(19) United States
(12) Patent Application Publication
(10) Pub. No.: US 2024/0325351 A1
(43) Pub. Date: Oct. 3, 2024

(54) METHOD OF ADMINISTRATION

(71) Applicant: Cybin UK Ltd., London (GB)

(72) Inventors: Peter RANDS, London (GB); Carol ROUTLEDGE, London (GB); Ellen JAMES, London (GB); Tiffanie BENWAY, London (GB); Zelah JOEL, London (GB); Victoria ATTWOOLL, London (GB); Meghan GOOD, London (GB)

(73) Assignee: Cybin UK Ltd., London (GB)

(21) Appl. No.: 18/619,547

(22) Filed: Mar. 28, 2024

Related U.S. Application Data
(60) Provisional application No. 63/585,000, filed on Sep. 25, 2023.
(60) Provisional application No. 63/513,140, filed on Jul. 12, 2023.
(60) Provisional application No. 63/493,428, filed on Mar. 31, 2023.

(51) Int. Cl. A61K 31/4045; A61K 9/00; A61K 45/06; A61P 25/24

(52) U.S. Cl. CPC A61K 31/4045; A61K 45/06; A61P 25/24; A61K 9/0019; A61K 9/0053

(57) ABSTRACT

The invention relates to a combination, combination for use, method for treating, kit, dosage regime, delivery device, method of adjuvant treatment, short-duration psychedelic agent for use, or parenteral formulation for use in the treatment of a psychiatric disorder in a patient. In particular, the invention relates to the administration of a short-duration psychedelic agent in combination with a monoamine antidepressant, such as a selective serotonin reuptake inhibitor (SSRI).

### FIELD OF THE INVENTION

The invention relates to a combination, combination for use, method for treating, kit for, dosage regime, delivery device, method of adjuvant treatment, short-duration psychedelic agent for use, or parenteral formulation for use in the treatment of a psychiatric disorder in a patient. In particular, the invention relates to the administration of a short-duration psychedelic agent in combination with a monoamine antidepressant agent, for example a selective serotonin reuptake inhibitor (SSRI).

### BACKGROUND OF THE INVENTION

Antidepressant agents are a broad class of pharmaceutical agents used to treat a number of psychiatric conditions, such as depression, anxiety, obsessive compulsive disorder, and eating disorders. Most regulator-approved antidepressant agents are monoamine antidepressant agents: non-psychedelic agents characterised by a mode of action which addresses one or more imbalance (usually a deficiency) of neurotransmitters serotonin, norepinephrine and dopamine. The standard of care in most applicable psychiatric conditions are selective serotonin reuptake inhibitors (SSRIs). SSRIs are widely used as antidepressants, generally used as the first-line treatment, and are thought to work by increasing serotonin levels in the brain. SSRIs are usually taken orally, typically in tablet form, and usually need to be taken daily for 2 to 4 weeks before any benefit is felt. SSRIs include citalopram, dapoxetine, escitalopram, fluoxetine, fluvoxamine, paroxetine, sertraline, and vortioxetine.

Large meta-analyses have shown that SSRIs are typically effective in approximately one third of patients within three months, a further third of patients respond within 12 months. The remaining third of patients do not respond and will typically remain on one or more antidepressant agent for many years without ever achieving a clinically meaningful response. Moreover, patient withdrawal from monoamine antidepressant agents such as SSRIs can be difficult and should be done slowly, with the dose reduced over a period of 4 weeks or more to prevent withdrawal symptoms (discontinuation syndrome). With over 300 million people suffering from depression worldwide, there remains a prevalent medical need to increase the clinical response rate of SSRIs and other monoamine antidepressant agents.

Classical psychedelics have also shown preclinical and clinical promise in treating psychiatric disorders (Carhart-Harris and Goodwin, Neuropsychopharmacology 42, 2105-2113 (2017)). For example, psilocybin has demonstrated significant improvement in a range of depression and anxiety rating scales in randomised double-blind studies (Griffiths et al. Journal of Psychopharmacology, 30(12), 1181-1197 (2016)). Efficacy of psilocybin has been shown in depression (R. L. Carhart-Harris et al., Psychopharmacology, 2018, 235, 399-408), end of life anxiety (R. R. Griffiths et al., J. Psychopharmacol., 2016, 30, 12, 1181-1197) and addiction (M. W. Johnson, A. Garcia-Romeu and R. R. Griffiths, Am. J. Drug Alcohol Abuse, 2017, 43, 1, 55-60), and is currently being investigated for several other mental health disorders that are rooted in psychologically destructive patterns of thought processing (Anorexia Nervosa: NCT #NCT04052568).

Becker et al. conducted a double-blind, placebo-controlled, crossover design clinical trial (clinicaltrials.gov identifier NCT03912974) to investigate the response to psilocybin (25 mg) in healthy volunteers after pre-treatment with escitalopram or placebo. Becker et al. report that daily escitalopram treatment for 2 weeks was not found to reduce the positive mood or mind-altering effects of a full dose of psilocybin in healthy subjects (Clin Pharmacol Ther. 2022 April; 111(4): 886-895. Published online 2021 Nov. 22. doi: 10.1002/cpt.2487). An open-label clinical trial has also been conducted to investigate the effects of orally administered psilocybin (wherein the duration of the psychedelic experience is typically six to eight hours long) in participants with treatment resistant depression, comparing participants who were receiving treatment with an SSRI with participants who had withdrawn from their SSRI treatment prior to the trial. The sponsor of the trial, Compass Pathways, announced that patients taking psilocybin with concomitant SSRIs showed ‘comparable treatment outcomes to patients withdrawn from their SSRI therapy’ (Compass Pathways Press Release dated 13 Dec. 2021: https://compasspathways.com/positive-outcome-25 mg-comp360-psilocybin-therapy-adjunct-ssri-antidepressants-open-label-treatment-resistant-depression-study/)).

Cybin Inc. have completed the Phase 1 part of a Phase 1/2a clinical trial evaluating a deuterated psilocybin analogue (CYB003) in healthy participants with and without major depressive disorder (ClinicalTrials.gov Identifier: NCT05385783). In February 2023, Cybin Inc. announced that following a single oral dose of CYB003, psychedelic effects were seen within ≈15 minutes and the average duration of peak effects lasted≈2 hours, data based on an interim analysis of CYB003 in healthy volunteers (https://cybin.com/cyb003/).

The University of California is conducting a Phase 1 clinical trial to compare the physiological and psychological effects of psilocin taken orally by pill or sublingually by dissolving a tablet under the tongue to those of psilocybin taken by pill in healthy adults (ClinicalTrials.gov Identifier: NCT05317689). Each participant will be administered 15.5 mg oral psilocin, 25 mg oral psilocybin, or 2.18 mg-4.36 mg sublingual psilocin.

Reunion Neuroscience have completed a Phase 1 clinical trial of a compound which is composed of a glutarate moiety appended to the phenolic functional group of 4-hydroxy-N,N-diisopropyltryptamine. The compound is stated to have a mean duration of the subjective experience at 33 mg of 3.7 hours (poster presented at American Society of Clinical Psychopharmacology Annual Meeting May 30-Jun. 2, 2023: https://reunionneuro.com/wp-content/uploads/2023/06/23-RNR-3851_ASCP23_RE104_Poster_M1-3.pdf). The compound N,N diisopropyltryptamine-4-glutarate, is also referred to as iprocin-4-glutarate, isoprocin-4-glutarate, or 4-glutarate-DIPT.

Short-duration psychedelic agents, for example N,N-dimethyltryptamine (DMT), have been shown to hold therapeutic value as rapid-acting antidepressants with durability of three months or more (Small Pharma Inc Press Release dated 25 Jan. 2023: https://smallpharma.com/press-releases/positive-topline-results-from-phase-iia-trial-of-sp1026-in-major-depressive-disorder/).

To-date no one has investigated the safety, tolerability or efficacy of short-duration psychedelics in patients receiving treatment with monoamine antidepressant agents such as SSRIs. The majority of clinical trials testing the efficacy of psychedelics in treating mental health disorders exclude patients currently taking SSRI medication. As both SSRI and serotonergic (or classical) psychedelics exert their primary effects through the serotonin system, theoretically, there are multiple ways in which these compounds could interact acutely:
a) Safety: SSRIs inhibit the reuptake of serotonin (5-hydroxytrytamine [5-HT]), meaning they increase intrasynaptic serotonin and therefore, increase activity at 5-HT receptors. Since classical psychedelics work by activating 5-HT receptors, these two activities together could risk inducing serotonin toxicity.
b) Pharmacodynamics/Efficacy: long-term use of SSRIs results in a homeostatic downregulation of 5-HT receptors, meaning that when a classical psychedelic is administered, the psychological effect may be blunted.
c) Pharmacodynamics/Efficacy: analysis of data from trials assessing the efficacy of MDMA-assisted therapy following discontinuation of SSRIs suggests that patients who have recently discontinued SSRI treatment may experience reduced efficacy of later serotonergic treatments (Feduccia, A. A., Jerome, L., Mithoefer, M. C., Holland, J. Discontinuations of medications classified as reuptake inhibitors affects treatment response of MDMA assisted psychotherapy. Psychopharmacology (Berl). 2021; 238: 581-588).

The present invention addresses these clinical uncertainties, and provides evidence of a surprising synergistic effect when a short-duration psychedelic agent is used in in combination with a monoamine antidepressant agent, including methods for treating, kits, or dosage regimes for use in the treatment of a psychiatric disorder in a patient.

### SUMMARY OF THE INVENTION

As a first aspect, the invention provides a combination comprising:
(i) a monoamine antidepressant agent; and
(ii) a short-duration psychedelic agent.

As a second aspect, the invention provides a method for treating a psychiatric disorder in a patient, preferably wherein the psychiatric disorder is selected from at least one of a depressive disorder, an anxiety disorder, obsessive compulsive disorder, and an eating disorder;
the method for treating comprising the administration to the patient of:
(i) a monoamine antidepressant agent; and
(ii) a short-duration psychedelic agent.

As a third aspect, the invention provides a kit comprising:
(i) a dosage form of a short-duration psychedelic agent; and
(ii) instructions for administration of the dosage form of a short-duration psychedelic agent to a patient in conjunction with a monoamine antidepressant agent.

As a fourth aspect, the invention provides a dosage regime, comprising:
(i) administration of a monoamine antidepressant agent; and
(ii) administration of a short-duration psychedelic agent.

As a fifth aspect, the invention provides a method of adjuvant treatment of a psychiatric disorder in a patient receiving treatment with a monoamine antidepressant agent, preferably wherein the psychiatric disorder is selected from at least one of a depressive disorder, an anxiety disorder, obsessive compulsive disorder, and an eating disorder; the method of adjuvant treatment comprising the administration to the patient of a short-duration psychedelic agent.

As a sixth aspect, the invention provides a short-duration psychedelic agent for use in the adjuvant treatment of a psychiatric disorder in a patient receiving treatment with a monoamine antidepressant agent, preferably wherein the psychiatric disorder is selected from at least one of a depressive disorder, an anxiety disorder, obsessive compulsive disorder, and an eating disorder.

As a seventh aspect, the invention provides the use of a short-duration psychedelic agent for the manufacture of a medicament for the adjuvant treatment of a psychiatric disorder in a patient receiving treatment with a monoamine antidepressant agent, preferably wherein the psychiatric disorder is selected from at least one of a depressive disorder, an anxiety disorder, obsessive compulsive disorder, and an eating disorder.

As an eighth aspect, the invention provides a parenteral formulation comprising a dose of a short-duration psychedelic agent for use in the treatment of a psychiatric disorder in a patient receiving treatment with a monoamine antidepressant agent; wherein the treatment comprises the administration, preferably parenteral administration, to the patient of the dose of the short-duration psychedelic agent; preferably wherein the psychiatric disorder is selected from at least one of a depressive disorder, an anxiety disorder, obsessive-compulsive disorder, and an eating disorder, preferably selected from a depressive disorder and an anxiety disorder.

As a ninth aspect, the invention provides a delivery device for use in the treatment of a psychiatric disorder in a patient receiving treatment with a monoamine antidepressant agent, wherein the delivery device is configured to deliver the administration, preferably parenteral administration, to the patient of a parenteral dose of a short-duration psychedelic agent.

For the avoidance of doubt, embodiments related to each aspect of the invention apply mutatis mutandis to the other aspects of the invention. Further aspects and embodiments of the present invention will be evident from the discussion herein.

In the first, second, third, fourth, fifth, sixth, seventh, eighth, and ninth aspect of the present invention the short-duration psychedelic agent is preferably selected from N,N-dimethyltryptamine, deuterated N,N-dimethyltryptamine, 5-methoxy-N,N-dimethyltryptamine, deuterated 5-methoxy-N,N-dimethyltryptamine, psilocin, deuterated psilocin, psilocybin, deuterated psilocybin, psilocin-4-glutarate, deuterated psilocin-4-glutarate, iprocin-4-glutarate, deuterated iprocin-4-glutarate, mixtures thereof, and pharmaceutically acceptable salts thereof.

In the first, second, third, fourth, fifth, sixth, seventh, eighth, and ninth aspect of the present invention the monoamine antidepressant agent is preferably a selective serotonin reuptake inhibitor (SSRI) or serotonin-norepinephrine reuptake inhibitor (SNRI).

### Definitions

Throughout this specification, one or more aspects of the invention may be combined with one or more features described in the specification to define distinct embodiments of the invention.

In the detailed description, reference is made to a number of terms, which are to be understood to have the meanings provided below, unless a context expressly indicates to the contrary.

The nomenclature used herein for the short-duration psychedelic agents for use in the invention is as commonly used in the field. The compounds described herein, may also be referred to by nomenclature in accordance with the rules of the International Union of Pure and Applied Chemistry (IUPAC) for chemical compounds, specifically the ‘IUPAC Compendium of Chemical Terminology (Gold Book)’ (see A. D. Jenkins et al., Pure & Appl. Chem., 1996, 68, 2287-2311). For the avoidance of doubt, if a rule of the IUPAC organisation is contrary to a definition provided herein, the definition herein is to prevail.
N,N-dimethyltryptamine (DMT) is also known by the IUPAC name 2-(1H-indol-3-yl)-N,N-dimethylethanamine, CAS Registry Number 61-50-7.
5-Methoxy-N,N-dimethyltryptamine (5-MeO-DMT) is also known by the IUPAC name 2-(5-methoxy-1H-indol-3-yl)-N,N-dimethylethanamine, CAS Registry Number 1019-45-0.
Psilocybin is also known by the IUPAC name 3-[2-(Dimethylamino)ethyl]-1H-indol-4-yl dihydrogen phosphate, CAS Registry Number 520-52-5.
Psilocin is also known by the IUPAC name 3-[2-(Dimethylamino)ethyl]-1H-indol-4-ol, CAS Registry Number 520-53-6.
N,N-diisopropyltryptamine (DIPT) is also known by the IUPAC name N-[2-(1H-indol-3-yl)ethyl]-N-propan-2-ylpropan-2-amine.
Iprocin is also known as 4-Hydroxy-N,N-diisopropyltryptamine or by IUPAC name 3-[2-(Diisopropylamino)ethyl]-1H-indol-4-ol, CAS Registry Number 132328-45-1.

The ‘compound of formula I’ described herein is a compound, or a pharmaceutically acceptable salt thereof, of the formula shown below:

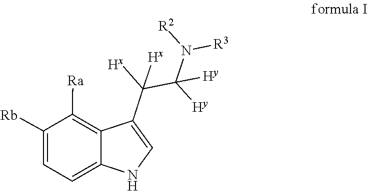

wherein:
Ra is selected from H, and D; or
Ra is selected from —OH, —OPO₃H₂ and —OC(O)C₁-C₃alkyleneCO₂H;
Rb is selected from H, D, —OCH₃ and —OCD₃;
R² and R³ are each independently selected from C(H^{z})₃; or
R² and R³ are each independently selected from C₂(H_{z})₅ and C₃(H^{z})₇; and
   each H^{x}, H^{y} and H^{z} is independently selected from protium and deuterium.

As used herein the terms ‘deuterated N,N-dimethyltryptamine,’ ‘deuterated 5-methoxy-N,N-dimethyltryptamine,’ ‘deuterated psilocin,’ ‘deuterated psilocybin,’ ‘deuterated N,N-diisopropyltryptamine’, deuterated psilocin-4-glutarate, and deuterated iprocin-4-glutarate, collectively ‘deuterated analogues’ of the short-duration psychedelic agent means that one or more hydrogen atom(s) in the structure of the short-duration psychedelic agent is replaced with a deuterium atom, wherein a deuterium atom is a hydrogen atom with an additional neutron. Substitution with deuterium may be at one or more of the α and β positions, on the alkyl groups represented by R² and R³ in the formulae below (such as methyl groups), on the indole ring, and/or in the methoxy group of 5-methoxy-N,N-dimethyltryptamine. In some embodiments, deuteration may be at the
α positions,
the α positions, and the β positions,
the alkyl groups represented by R² and R³ in the formulae below (e.g., methyl groups),
the alkyl groups represented by R² and R³ in the formulae below (e.g., methyl groups) and the α positions,
the alkyl groups represented by R² and R³ in the formulae below (e.g., methyl groups) and the α positions, and the 0 positions.

Where compounds described herein are indicated as being or described as deuterated or as deuterated analogues, the compound concerned is enriched with deuterium by an amount that is dependent on the percentage of deuterium available in the reagents from which the compounds are derived. For example, the d6-dimethylamino portions of compounds of formula I, wherein —NR²R³ is —N(CD₃)₂, may be derived from dimethyl-d7-amine, or dimethyl-d6-amine (commonly available as HCl salts), which are available from chemical vendors in purities of deuterium that range from 98% to 99%. The purity of deuterium in the resultant d6-dimethylamino substituents is consequently between 98% and 99%. This means, as the skilled person will understand, that not all compounds of formula I (for example) will comprise d6-dimethylamino substituents—some may comprise d0-d5-dimethylamino, but the average purity of deuterium is about 98% to 99%. It will be understood that the term deuterated analogues includes isotopic mixtures of these compounds, for example an isotopic mixture of d0, d1 and d2-deuterated N,N-dimethyltryptamine or deuterated 5-methoxy-N,N-dimethyltryptamine. Such isotopic mixtures are described in published patent applications WO2022/117359 and WO2020/245133, the disclosures of which are hereby incorporated by reference in their entireties.

In some embodiments, the short-duration psychedelic agent is substituted at position 5 with methoxy. The term ‘methoxy’ (often abbreviated to OMe) defines a univalent group derived from methanol by removal of a hydrogen atom from the OH moiety.

In some embodiments, the short-duration psychedelic agent is substituted at position 4 with —OH, —OPO₃H₂ or —OC(O)C₁-C₃alkyleneCO₂H, preferably —OPO₃H₂ or glutarate (—OC(O)C₃alkyleneCO₂H). The term ‘C₁-C₃alkylene’ refers to a linear saturated divalent hydrocarbon radical of one, two, or three carbon atoms, i.e., methylene, ethylene, or propylene.

For the avoidance of doubt, position 5 of the short-duration psychedelic agent refers to the positions labelled in the structure below (substitution not shown), wherein R² and R³ are both methyl, or as defined in any one of the embodiments below.

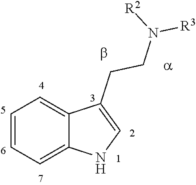

References herein to the dose or total dose of a short-duration psychedelic agent refer to the dose or total dose as the free base equivalent of that agent. For example, where the salt (such as the fumarate salt) of the short-psychedelic agent is used, the dose or total dose is specified as the equivalent dose of the free base.

References herein to an ‘antidepressant agent’ refer to an agent that is an approved pharmacological treatment for a depressive disorder in at least one jurisdiction, such as the United States of America, the European Union, the United Kingdom, Australia, New Zealand, and Japan. References herein to a ‘monoamine antidepressant agent’ refer to a non-psychedelic agent characterised by a mode of action which addresses one or more imbalance of neurotransmitters selected from serotonin, norepinephrine and dopamine.

As a proviso, the monoamine antidepressant agent is preferably not a monoamine oxidase-inhibitor class antidepressant. At certain dosage levels, monoamine oxidase-inhibitor antidepressants may interfere with and extend the duration of the short-duration psychedelic. Preferred monoamine antidepressant agents for use in the present invention are serotonin reuptake inhibitors, such as selective serotonin reuptake inhibitors (SSRIs) and serotonin-norepinephrine reuptake inhibitors (SNRIs).

Serotonin-norepinephrine reuptake inhibitors (SNRIs) are a class of monoamine antidepressant agents which inhibit the reuptake of serotonin and norepinephrine, and which are used to treat a number of disorders including depressive disorders, anxiety disorders and obsessive-compulsive disorder (OCD). SNRIs approved by the Food and Drug Administration (FDA) in the US include desvenlafaxine, duloxetine, levomilnacipran, milnacipram, and venlafaxine.

Selective serotonin reuptake inhibitors (SSRIs) are a class of monoamine antidepressant agents which selectively inhibit the reuptake of serotonin and are used to treat a number of disorders including depressive disorders, anxiety disorders, obsessive-compulsive disorder (OCD) and post-traumatic stress disorder (PTSD). In the United Kingdom, SSRIs are recommended as the first line treatment for depressive disorders. Fluoxetine is approved by the FDA in the US and by the EMA in Europe for the treatment of bulimia nervosa. Approved SSRIs include citalopram, escitalopram, fluoxetine, fluvoxamine, paroxetine, sertraline, dapoxetine, and vortioxetine.

The term ‘in conjunction with’ as used herein refers to treatment with a short-duration psychedelic administered to a patient receiving treatment with a monoamine antidepressant agent. This may also be referred to as ‘adjuvant treatment’, ‘adjunctive treatment’ or ‘combined treatment.’ Typically, the short-duration psychedelic agent is administered to a patient who has received an adequate course of one or more antidepressant agents, and is continuing to receive treatment with a monoamine antidepressant agent, i.e., the patient receives ongoing treatment with the monoamine antidepressant agent before and during treatment with the short-duration psychedelic agent. An adequate course of a monoamine antidepressant agent will vary according to the specific antidepressant agent, but will typically be a course of treatment over a period of 4 weeks or more, preferably a period of 6 weeks or more. For example, the patient may take a once (or twice or more) a day dose of a monoamine antidepressant agent over a period of 4 weeks or more, preferably a period of 6 weeks or more. Typically, the patient has not experienced a clinically meaningful improvement in symptoms following administration of an adequate course of treatment with the monoamine antidepressant agent. The term ‘in conjunction with’ as used herein does not require simultaneous administration with the monoamine antidepressant agent and the short-duration psychedelic agent. For the avoidance of doubt, the monoamine antidepressant agent and the short-duration psychedelic agent may be administered and/or prescribed by different healthcare practitioners, such as different physicians. Typically, the monoamine antidepressant agent and the short-duration psychedelic agent are administered separately, and may be administered sequentially. Optionally, the monoamine antidepressant agent and the short-duration psychedelic agent may be administered simultaneously. Typically the monoamine antidepressant agent is administered daily over the period of a course of treatment, while the short-duration psychedelic agent is administered as a single dose.

As used herein, the term ‘synergy’ or ‘synergistic’ refers to an effect that is greater than additive.

As used herein the term ‘parenteral administration’ or ‘parenterally administering’ means the administration of a dose of a short-duration psychedelic agent via any route other than oral, in one or more portions over the duration of the dosing period. The parenteral administration is preferably essentially continuous for a dosing period of up to about 15 minutes, such as from about 5 to about 15 minutes, or from about 5 to about 15 minutes, or from about 8 to about 12 minutes, or from about 9 to about 11 minutes, or from about 6 to about 14 minutes, or from about 7 to about 13 minutes, or from about 8 to about 11 minutes, or about 9 minutes, or about 10 minutes, or about 11 minutes. In other embodiments, the parenteral administration may comprise an initial bolus administration of a short-duration psychedelic agent, that is a single dose of an agent administered at a rapid rate, typically over about 30 to about 60 seconds, which single dose provides a fast onset breakthrough psychedelic experience, followed by an essentially continuous administration for a period of from about 5 to about 15 minutes. In other embodiments, the parenteral administration may comprise a bolus administration of a psychedelic agent, that is a single dose of an agent administered at a rapid rate, typically over about 30 to about 60 seconds. In some embodiments, the administration comprises a bolus, such as intramuscular, intranasal or intravenous bolus administration.

As used herein the term ‘effective dose’ refers to a dose which is sufficient to elicit a psychedelic experience, i.e., a period in which the patient experiences one or more of intense reactions or emotions, altered state of perception, visual or other sensory hallucinations, spiritual experience, ego dissolution and dissociation. Ego dissolution describes a state in which the boundary between an individual and the outside world dissolves. Dissociation describes a state in which an individual experiences a sensation that different parts of the brain are not connected, such as a disconnect between mind and body.

As used herein, the term ‘therapeutically effective dose’ refers to a dose of the monoamine antidepressant agent and the short-duration psychedelic agent which, in combination, provide therapeutic efficacy. The dose of each separate agent may not individually provide therapeutic efficacy.

A ‘month’ as used herein is defined as a calendar month.

‘Administration occurs only one time in any three-month period’ as used herein means an interval of at least 90 days is present between administrations. Likewise, ‘administration occurs only one time in any six-month period’ as used herein means an interval of at least 180 days is present between administrations, and so on.

‘Administration occurs at most once every three months’ means at least three months, i.e. 90 days pass between administrations. Likewise, ‘administration occurs at most once every six months’ means at least six months, i.e. 180 days pass between administrations, and so on.

References herein to a singular of a noun encompass the plural of the noun, and vice-versa, unless the context implies otherwise.

Throughout this specification the word ‘comprise’, or variations such as ‘comprises’ or ‘comprising’, will be understood to imply the inclusion of a stated element, integer or step, or group of elements, integers or steps, but not the exclusion of any other element, integer or step, or group of elements, integers or steps. The term ‘comprising’ includes within its ambit the term ‘consisting’ or ‘consisting essentially of’.

The term ‘consisting’ or variants thereof is to be understood to imply the inclusion of a stated element, integer or step, or group of elements, integers or steps, and the exclusion of any other element, integer or step or group of elements, integers or steps.

The term ‘consisting essentially of’ or variants thereof is to be understood to imply the inclusion of a stated element, integer or step, or group of elements, integers or steps, and that further components may be present, but only those not materially affecting the essential characteristics of the embodiment of the invention.

The term ‘about’ herein, when qualifying a number or value, is used to refer to values that lie within ±5% of the value specified. For the avoidance of doubt, where a number or value is specified herein in the absence of the term ‘about’, the number or value should be understood according to standard numeric rounding conventions according to the number of decimal places. For example, a whole number, such as 6, is understood to encompass values ≥5.5 and <6.5. Likewise, a number specified to one decimal place, such as 5.3, is understood to encompass values ≥5.25 and <5.35.

Where a range of values is provided, the range includes the end point values. For example, the range 20 to 28, or from 20 to 28, would include the values 20, 21, 22, 23, 24, 25, 26, 27 and 28; the range 5 to 15, or from 5 to 15, would include the values 5, 6, 7, 8, 9, 10, 11, 12, 13, 14 and 15.

As used herein, the term ‘patient’ preferably refers to a mammal. Typically, the mammal is a human, but may also refer to a domestic mammal. The term does not encompass laboratory mammals.

As used herein, the term ‘in combination with psychotherapy’ or ‘psychedelic assisted psychotherapy’ refers to the treatment of a psychiatric disorder by psychological means, which are enhanced by the combination, method of treatment, or kit of the invention. The psychotherapeutic practice is provided alongside a dose of the short-duration psychedelic agent, including for example any dose as defined in the present invention.

The term ‘treatment’ defines the therapeutic treatment of a patient, in order to reduce or halt the rate of progression of a disorder, or to ameliorate or cure the disorder. Prophylactic treatment of a patient having a diagnosed psychiatric disorder is also included. Such prophylactic treatment, also referred to as secondary prevention, aims to reduce the impact of the disorder and/or to hinder development of the disorder through treatment in accordance with the invention.

As used herein, the term ‘improvement’ refers to a clinically meaningful reduction in symptom severity over a period of time, compared to a baseline assessment prior to administration of the short-duration psychedelic agent, or as assessed by patient reported outcomes. Improvement may be assessed by patient reported outcomes or clinician reported outcomes, such as a structured patient interview or questionnaire. The term ‘clinician’ refers to a qualified and/or registered doctor or healthcare professional, including therapists, psychiatrists and psychologists.

Patient reported outcomes or clinician reported outcomes may comprise assessments using rating scales, which include, but are not limited to, the Clinical Global Impression scale (CGI), an observer rated scale consisting of one or more of three different measures—severity of illness (CGI-S), global improvement (CGI-I) and the efficacy index (CGI-E); the Montgomery-Asberg Depression Rating Scale (MADRS), a 10-point diagnostic questionnaire; the 17-item Hamilton Depression Rating Scale (HAMD17); the Beck Depression Inventory-II; the Ruminative Response Scale (RRS-22), a self-reported measure consisting of 22 items and three factors—depression, brooding and reflection; the Beck Anxiety Inventory (BAI); the Hamilton Anxiety Scale (HAM-A); the State-Trait Anxiety Inventory (STAI or STAI-T); Generalised Anxiety Disorder Assessment (GAD-7); the Clinician-Administered Posttraumatic Stress Disorder Scale (CAPS); the Yale-Brown Obsessive Compulsive Scale; and suicidality rating scales such as the Beck Scale for Suicidal Ideation (BSS), Columbia-Suicide Severity Rating Scale (C-SSRS), and the suicidal thoughts item of the MADRS for suicidal ideation or the Clinical Global Impression of Severity of Suicidality-Revised (CGI-SS-R) scale. Patient reported outcomes may also include the Beck Depression Inventory (BDI-II); the Quality of Life Enjoyment and Satisfaction Questionnaire-Short Form (Q-LES-Q-SF), a 16-item self-administered questionnaire; the EQ-5D-5L descriptive system and EQ VAS; the Patient Global Impression of Severity scale (PGI-S); and the Patient Global Impression of Improvement scale (PGI-I). A combination of the rating scales may be used. Clinician or patient reported outcomes may comprise weight gain for anorexia nervosa; frequency of binge-purge episodes for bulimia nervosa, or frequency of binge episodes for binge eating disorder.

Measurement of a clinically meaningful reduction in symptom severity will be assessed in accordance with the rating scale or other clinician or patient reported outcomes, including:

Typically for the Clinical Global Impression scale (CGI), a response score of 0, 1, 2 or 3 indicates improvement, preferably 0, 1 or 2.

Typically, for rating scales such as the Montgomery-Asberg Depression Rating Scale (MADRS), Hamilton Depression Rating Scale (HAMD17), Beck Depression Inventory-II (BDI-II), Beck Anxiety Inventory (BAI), Beck Scale for Suicidal Ideation (BSS), Columbia-Suicide Severity Rating Scale (C-SSRS), Hamilton Anxiety Scale (HAM-A), State-Trait Anxiety Inventory (STAI or STAI-T), Generalised Anxiety Disorder Assessment (GAD-7), Yale-Brown Obsessive Compulsive Scale, Quality of Life Enjoyment and Satisfaction Questionnaire-Short Form (Q-LES-Q-SF); EQ-5D-5L descriptive system and EQ VAS, and Clinician-Administered Posttraumatic Stress Disorder Scale (CAPS); a 15% or greater reduction in the score compared with baseline assessment indicates improvement, preferably 20% or greater, or 30% or greater, or 50% or greater.

Alternatively, clinically meaningful reduction in symptom severity may be assessed by the point reduction on the rating scale. For example, improvement is indicated by the following point reductions: for the MADRS scale, a point reduction of 6 or greater; for the HAMD-17 scale, a point reduction of 4 or greater; for the Beck Depression Inventory-II (BDI-II) scale, a point reduction of 12 or greater. Alternatively, a reduction in the severity category indicates improvement, for example from severe to moderate.

Typically for the Columbia-Suicide Severity Rating Scale (C-SSRS), a reduction in the number of questions answered ‘yes’ indicates improvement. Alternatively, a reduction in the intensity category indicates improvement, for example a reduction from very severe to severe, or from severe to moderately severe.

Typically, for the Patient Global Impression of Severity scale (PGI-S), a reduction in the severity rating assessed by the patient indicates improvement, e.g., a reduction in the severity rating from 6 (severely ill) to 4 (moderately ill).

Typically for the Patient Global Impression of Improvement scale (PGI-I), a rating of 3 (minimally improved) or below indicates improvement, preferably a score of 2 (much improved) or below. The PGI-I scale is not assessed in comparison with the baseline assessment, but is a post-dosing only assessment.

As used herein, the term ‘remission’ refers to a reduction in the symptoms of the psychiatric disorder to a level which indicates mild symptoms, or that the patient is in the healthy range, i.e., the patient is not experiencing symptoms indicative of a clinical condition. For depressive disorders, remission is typically considered to be a score of 13 or below on the MADRS rating score, preferably 10 or below; or 7 or below on the HAMD17 rating scale; or 19 or below on the Beck Depression Inventory—II scale; or 15 or below on the Beck Anxiety Inventory scale; or 17 or below on the Hamilton Anxiety scale; or 15 or below on the Yale Brown Obsessive Compulsive scale; or 3 or below on the Patient Global Impression-Severity Scale (PGI-S). Remission is typically considered to apply to a patient who answers “no” to each question on the Columbia-Suicide Severity Rating Scale (C-SSRS), or a score of 10 or below (moderate), preferably 6 or below.

As used herein, the term ‘short-duration psychedelic agent’ or ‘short-duration psychedelic’ refers to a psychedelic agent which elicits a psychedelic experience with a duration of 4 hours or less, or 3 hours or less, or 2 hours or less, or 1 hour or less. Preferably, the short-duration psychedelic elicits a psychedelic experience of 1 hour or less. Preferably the short-duration psychedelic is administered parenterally. Typical short-duration psychedelic agents are delivered parenterally, i.e., not administered orally, either because the agent is not orally active (e.g., DMT, 5-MeO-DMT) or because oral administration leads to a long-duration psychedelic experience. For example, oral administration of psilocybin elicits a duration of psychedelic experience of about 6 to 8 hours. However, certain orally active psychedelics (e.g., deuterated DMT or deuterated psilocybin) may elicit a psychedelic experience with a shorter duration, for example of 3 hours or less, preferably 1 hour or less. Hence, the present invention provides for a duration of psychedelic experience of 3 hours or less, preferably 1 hour or less, more preferably 45 minutes or less, yet more preferably 30 minutes or less, and, in preferred embodiments of any aspect or embodiment of the invention, the short-duration psychedelic agent is provided in a dosage form suitable for parenteral administration, preferably by injection, inhalation, insufflation, transdermal or transmucosal administration.

As used herein, the term ‘psychiatric disorder’ is characterised by a clinically significant disturbance in an individual's cognition, emotional regulation, or behaviour, and that is associated with present distress (e.g., a painful symptom) or disability (i.e., impairment in one or more important areas of functioning) or with a significantly increased risk of suffering death, pain, disability, or an important loss of freedom. Diagnostic criteria for psychiatric disorders referred to herein are provided in the Diagnostic and Statistical Manual of Mental Disorders, Fifth Edition, (DSM-5).

As used herein the term ‘obsessive-compulsive disorder’ (OCD) is defined by the presence of either obsessions or compulsions, but commonly both. The symptoms can cause significant functional impairment and/or distress. An obsession is defined as an unwanted intrusive thought, image or urge that repeatedly enters the person's mind. Compulsions are repetitive behaviours or mental acts that the person feels driven to perform. Typically, OCD manifests as one or more obsessions, which drive adoption of a compulsion. For example, an obsession with germs may drive a compulsion to clean, or an obsession with food may drive a compulsion to overeat, eat too little or throw up after eating (i.e., an obsession with food may manifest itself as an eating disorder). A compulsion can either be overt and observable by others, such as checking that a door is locked, or a covert mental act that cannot be observed, such as repeating a certain phrase in one's mind. The Yale-Brown Obsessive Compulsive Scale (Y-BOCS) rating scale may be used to assess OCD.

As used herein, the term ‘eating disorder’ is defined by severe and persistent disturbance in eating behaviours and associated distressing thoughts and emotions. The term ‘eating disorder’ includes, but is not limited to, anorexia nervosa and bulimia nervosa, binge eating disorder, avoidant restrictive food intake disorder, other specified feeding and eating disorder, pica and rumination disorder.

Eating disorders often co-occur with anxiety disorders and obsessive compulsive disorder. Neziroglu and Sandler differentiate between OCD and an eating disorder: ‘Whereas patients with eating disorders are primarily driven by concerns of physical appearance, and consequently alter their eating patterns in order to lose weight accordingly. OCD patients may be restricting their eating for reasons very diferent than body image concerns’ (https://iocdf.orgexpert-opinions/expert-opinion-eating-disorders-and-ocd/).

The term “eating disorder” includes anorexia nervosa, bulimia and binge eating disorder (BED). The symptoms of anorexia nervosa include eating too little and/or exercising too much in order to keep weight as low as possible. The symptoms of bulimia include eating a lot of food in a very short amount of time (i.e., binging) and then being deliberately sick, using laxatives, eating too little and/or exercising too much to prevent weight gain. The symptoms of BED include regularly eating large portions of food until uncomfortably full, and consequently feeling upset or guilty.

As used herein the term ‘depressive disorder’ includes, but is not limited to, major depressive disorder, persistent depressive disorder, bipolar disorder, bipolar depression, depression in terminally ill patients, and treatment resistant depression.

As used herein the term ‘major depressive disorder’ (MDD, also referred to as major depression or clinical depression) is defined as the presence of five or more of the following symptoms over a period of two-weeks or more (also referred to herein as a ‘major depressive episode’), most of the day, nearly every day:
depressed mood, such as feeling sad, empty or tearful (in children and teens, depressed mood can appear as constant irritability);
significantly reduced interest or feeling no pleasure in all or most activities;
significant weight loss when not dieting, weight gain, or decrease or increase in appetite (in children, failure to gain weight as expected);
insomnia or increased desire to sleep;
either restlessness or slowed behaviour that can be observed by others;
fatigue or loss of energy;
feelings of worthlessness, or excessive or inappropriate guilt;
trouble making decisions, or trouble thinking or concentrating;
recurrent thoughts of death or suicide, or a suicide attempt.

At least one of the symptoms must be either a depressed mood or a loss of interest or pleasure.

Persistent depressive disorder, also known as dysthymia, is defined as a patient exhibiting the following two features:
A. Has depressed mood for most of the time almost every day for at least two years. Children and adolescents may have irritable mood, and the time frame is at least one year.
B. While depressed, a person experiences at least two of the following symptoms:
Either overeating or lack of appetite.
Sleeping too much or having difficulty sleeping.
Fatigue, lack of energy.
Poor self-esteem.
Difficulty with concentration or decision-making.

As used herein, the term ‘treatment resistant major depressive disorder’ describes MDD that fails to achieve an adequate response to treatment with standard of care therapy.

As used herein, ‘bipolar disorder’, also known as manic-depressive illness, is a disorder that causes unusual shifts in mood, energy, activity levels, and the ability to carry out day-to-day tasks.

There are two defined sub-categories of bipolar disorder; both of them involve clear changes in mood, energy, and activity levels. These moods range from periods of extremely ‘up,’ elated, and energised behaviour (known as manic episodes, and defined further below) to very sad, ‘down,’ or hopeless periods (known as depressive episodes). Less severe manic periods are known as hypomanic episodes.

Bipolar I Disorder—defined by manic episodes that last at least 7 days, or by manic symptoms that are so severe that the person needs immediate hospital care. Usually, depressive episodes occur as well, typically lasting at least 2 weeks. Episodes of depression with mixed features (having depression and manic symptoms at the same time) are also possible.

Bipolar II Disorder—defined by a pattern of depressive episodes and hypomanic episodes, but not the full-blown manic episodes described above.

As used herein ‘bipolar depression’ is defined as an individual who is experiencing depressive symptoms with a previous or coexisting episode of manic symptoms, but does not fit the clinical criteria for bipolar disorder.

Depressive disorders are typically assessed using a rating scale selected from Clinical Global Impression scale (CGI); the Montgomery-Asberg Depression Rating Scale (MADRS); Hamilton Depression Rating Scale (HAMD-17); the Beck Depression Inventory-II; the Beck Anxiety Inventory (BAI); suicidality rating scales such as the Beck Scale for Suicidal Ideation (BSS), Columbia-Suicide Severity Rating Scale (C-SSRS), the suicidal thoughts item of the MADRS for suicidal ideation, the Clinical Global Impression of Severity of Suicidality-Revised (CGI-SS-R) scale, or the Columbia-Suicide Severity Rating Scale (C-SSRS); and combinations of these scales. Depressive disorders may also be assessed using patient reported outcomes selected from the Beck Depression Inventory (BDI-II); the Quality of Life Enjoyment and Satisfaction Questionnaire-Short Form (Q-LES-Q-SF); the EQ-5D-5L descriptive system and EQ VAS; the Patient Global Impression of Severity scale (PGI-S); and combinations of these outcome assessments.

Depressive disorders are preferably assessed using the Columbia-Suicide Severity Rating Scale (C-SSRS), the Beck Depression Inventory-II, the Beck Scale for Suicidal Ideation (BSS), the Montgomery-Asberg Depression Rating Scale (MADRS), and/or the 17-item Hamilton Depression Rating Scale (HAMD-17).

As used herein, the term ‘anxiety disorder’ includes, but is not limited to, generalised anxiety disorder, phobia, panic disorder, social anxiety disorder, and post-traumatic stress disorder. Anxiety disorders are typically assessed using a rating scale selected from the Beck Anxiety Inventory (BAI), Hamilton Anxiety Scale (HAM-A), State-Trait Anxiety Inventory (STAI or STAI-T), Generalised Anxiety Disorder Assessment (GAD-7), and combinations of these scales. Anxiety disorders may also be assessed using patient reported outcomes, as described above.

‘Generalised anxiety disorder’ (GAD) as used herein means a chronic disorder characterised by long-lasting anxiety that is not focused on any one object or situation. Those suffering from GAD experience non-specific persistent fear and worry, and become overly concerned with everyday matters. GAD is characterised by chronic excessive worry accompanied by three or more of the following symptoms: restlessness, fatigue, concentration problems, irritability, muscle tension, and sleep disturbance.

‘Phobia’ is defined as a persistent fear of an object or situation the affected person will go to great lengths to avoid, typically disproportional to the actual danger posed. If the feared object or situation cannot be avoided entirely, the affected person will endure it with marked distress and significant interference in social or occupational activities.

A patient suffering from a ‘panic disorder’ is defined as one who experiences one or more brief attack (also referred to as a panic attack) of intense terror and apprehension, often marked by trembling, shaking, confusion, dizziness, nausea, and/or difficulty breathing. A panic attack is defined as a fear or discomfort that abruptly arises and peaks in less than ten minutes.

‘Social anxiety disorder’ is defined as an intense fear and avoidance of negative public scrutiny, public embarrassment, humiliation, or social interaction. Social anxiety often manifests specific physical symptoms, including blushing, sweating, and difficulty speaking.

‘Post-traumatic stress disorder’ (PTSD) is an anxiety disorder that results from a traumatic experience. Post-traumatic stress can result from an extreme situation, such as combat, natural disaster, rape, hostage situations, child abuse, bullying, or even a serious accident. Common symptoms include hypervigilance, flashbacks, avoidant behaviours, anxiety, anger and depression.

The Clinician-Administered Posttraumatic Stress Disorder Scale (CAPS) may be used to assess PTSD symptoms.

As used herein, the term ‘post-partum depression’ (PPD, also known as postnatal depression) is a form of depression experienced by either parent of a newborn baby. Symptoms typically develop within 4 weeks of delivery of the baby and often include extreme sadness, fatigue, anxiety, loss of interest or pleasure in hobbies and activities, irritability, and changes in sleeping or eating patterns.

### DETAILED DESCRIPTION

The combination, method for treating, kit, dosage regime, delivery device, parenteral formulation, or method of adjuvant treatment of the invention is based on the surprising discovery that the administration of a single, effective dose of a short-duration psychedelic such as N,N-dimethyltryptamine to a patient being treated with a monoamine antidepressant agent provided a greater improvement in symptoms when compared with administration of the short-duration psychedelic agent in the absence of monoamine antidepressant agent treatment, such as SSRI treatment. The greater improvement in symptoms may provide a number of advantages. The advantages may include an improved response rate, an improved remission rate, reduced dose of one or both agents, and reduction in use of, or more effective withdrawal for the monoamine antidepressant agent following administration of the short-duration psychedelic agent.

Accordingly, the first aspect of the invention provides, as embodiment 1, a combination comprising:
(i) A monoamine antidepressant agent; and
(ii) a short-duration psychedelic agent.

The second aspect of the invention provides, as embodiment 2, a method for treating a psychiatric disorder in a patient, preferably wherein the psychiatric disorder is selected from at least one of a depressive disorder, an anxiety disorder, obsessive compulsive disorder, and an eating disorder, the method for treating comprising the administration to the patient:
(i) a monoamine antidepressant agent; and
(ii) a short-duration psychedelic agent.

The third aspect of the invention provides, as embodiment 3, a kit comprising:
(i) a dosage form of a short-duration psychedelic agent; and
(ii) instructions for administration of the dosage form of a short-duration psychedelic agent to a patient in conjunction with a monoamine antidepressant agent.

The fourth aspect of the invention provides, as embodiment 4, a dosage regime comprising:
(i) administration of a monoamine antidepressant agent; and
(ii) administration of a short-duration psychedelic agent.

The fifth aspect of the invention provides, as embodiment 5, a method of adjuvant treatment of a psychiatric disorder in a patient receiving treatment with a monoamine antidepressant agent, preferably wherein the psychiatric disorder is selected from at least one of a depressive disorder, an anxiety disorder, obsessive compulsive disorder, and an eating disorder; the method of adjuvant treatment comprising the administration to the patient of a short-duration psychedelic agent.

The sixth aspect of the invention provides, as embodiment 6, a short-duration psychedelic agent for use in the adjuvant treatment of a psychiatric disorder in a patient receiving treatment with a monoamine antidepressant agent, preferably wherein the psychiatric disorder is selected from at least one of a depressive disorder, an anxiety disorder, obsessive compulsive disorder, and an eating disorder.

The invention further provides, as embodiment 7, a combination according to embodiment 1 wherein the combination is synergistic; or a method for treating, kit, dosage regime, method of adjuvant treatment, or short-duration psychedelic agent for use according to any one of embodiments 2 to 6, wherein the administration of
(i) a monoamine antidepressant agent; and
(ii) a short-duration psychedelic agent
   provides a synergistic effect.

The invention further provides, as embodiment 8, a combination, method for treating, kit, dosage regime, method of adjuvant treatment, or short-duration psychedelic agent for use according to any preceding embodiment, wherein the monoamine antidepressant agent is selected from a selective serotonin reuptake inhibitor and a serotonin-norepinephrine reuptake inhibitor.

The invention further provides, as embodiment 9, a combination, method for treating, kit, dosage regime, method of adjuvant treatment, or short-duration psychedelic agent for use according to any preceding embodiment, wherein the short-duration psychedelic agent is selected from N,N-dimethyltryptamine, deuterated N,N-dimethyltryptamine, 5-methoxy-N,N-dimethyltryptamine, deuterated 5-methoxy-N,N-dimethyltryptamine, psilocin, deuterated psilocin, psilocybin, deuterated psilocybin, psilocin-4-glutarate, deuterated psilocin-4-glutarate, iprocin-4-glutarate, deuterated iprocin-4-glutarate, mixtures thereof and pharmaceutically acceptable salts thereof, preferably the short-duration psychedelic agent is selected from N,N-dimethyltryptamine, deuterated N,N-dimethyltryptamine, 5-methoxy-N,N-dimethyltryptamine, and deuterated 5-methoxy-N,N-dimethyltryptamine, mixtures thereof and pharmaceutically acceptable salts thereof.

The invention further provides, as embodiment 10, a combination, method for treating, kit, dosage regime, method of adjuvant treatment, or short-duration psychedelic agent for use, according to any preceding embodiment, wherein the monoamine antidepressant agent is selected from at least one of desvenlafaxine, duloxetine, levomilnacipran, milnacipram, venlafaxine, citalopram, escitalopram, fluoxetine, fluvoxamine, paroxetine, sertraline, dapoxetine, and vortioxetine, and the pharmaceutically acceptable salts thereof.

The invention further provides, as embodiment 11, a combination according to any one of embodiments 1 to 10, comprising
(i) an adequate course of treatment of a monoamine antidepressant agent; and
(ii) a single dose of a short-duration psychedelic agent; or
a method for treating, kit, dosage regime, method of adjuvant treatment, or short-duration psychedelic agent for use, according to any preceding embodiment, comprising:
(i) administration of an adequate course of a monoamine antidepressant agent; and
(ii) administration of a single dose of a short-duration psychedelic agent.

The invention further provides, as embodiment 12, a combination, a method for treating, kit, dosage regime, method of adjuvant treatment, or short-duration psychedelic agent for use according to embodiment 11, wherein the adequate course of treatment comprises a period of treatment of 4 weeks or more.

The invention further provides, as embodiment 13, a combination, a method for treating, kit, dosage regime, method of adjuvant treatment, or short-duration psychedelic agent for use, according to any one of embodiments 11 and 12, wherein said administration of the single dose of a short-duration psychedelic agent occurs only one time in any three-month period, or wherein said single dose of a short-duration psychedelic agent is for administration only one time in any three-month period. As a further embodiment the invention provides a combination, a method for treating, kit, dosage regime, method of adjuvant treatment, or short-duration psychedelic agent for use, according to any one of embodiments 11 and 12, wherein said administration of the single dose of a short-duration psychedelic agent occurs at most once every three months, or wherein said single dose of a short-duration psychedelic agent is for administration at most once every three months.

The invention further provides, as embodiment 14, a method for treating, kit, dosage regime, method of adjuvant treatment, or short-duration psychedelic agent for use, according to any one of embodiments 11 and 12, wherein the administration of the single dose of short-duration psychedelic agent occurs only one time in any six-month period, or wherein said single dose of a short-duration psychedelic agent is for administration only one time in any six-month period; or a combination according to any one of embodiments 11 and 12, wherein the single dose of a short-duration psychedelic agent is for administration only one time in any six-month period. As a further embodiment the invention provides a combination, a method for treating, kit, dosage regime, method of adjuvant treatment, or short-duration psychedelic agent for use, according to any one of embodiments 11 and 12, wherein said administration of the single dose of a short-duration psychedelic agent occurs at most once every six months, or wherein said single dose of a short-duration psychedelic agent is for administration at most once every six months.

The invention further provides, as embodiment 15, a method for treating, kit, dosage regime, method of adjuvant treatment, or short-duration psychedelic agent for use, according to any one of embodiments 11 and 12, wherein said administration of the single dose of a short-duration psychedelic agent occurs only one time in any nine-month period, or wherein said single dose of a short-duration psychedelic agent is for administration only one time in any nine-month period; or a combination according to any one of embodiments 11 and 12, wherein the single dose of a short-duration psychedelic agent is for administration only one time in any nine-month period. As a further embodiment the invention provides a combination, a method for treating, kit, dosage regime, method of adjuvant treatment, or short-duration psychedelic agent for use, according to any one of embodiments 11 and 12, wherein said administration of the single dose of a short-duration psychedelic agent occurs at most once every nine months, or wherein said single dose of a short-duration psychedelic agent is for administration at most once every nine months.

The invention further provides, as embodiment 16, a combination, a method for treating, kit, dosage regime, method of adjuvant treatment, or short-duration psychedelic agent for use, according to any one of embodiments 11 and 12, wherein said administration of the single dose of a short-duration psychedelic agent occurs only one time in any twelve-month period, or wherein said single dose of a short-duration psychedelic agent is for administration only one time in any twelve-month period; or a combination according to any one of embodiments 11 and 12, wherein the single dose of a short-duration psychedelic agent is for administration only one time in any twelve-month period. As a further embodiment the invention provides a combination, a method for treating, kit, dosage regime, method of adjuvant treatment, or short-duration psychedelic agent for use, according to any one of embodiments 11 and 12, wherein said administration of the single dose of a short-duration psychedelic agent occurs at most once every twelve months, or wherein said single dose of a short-duration psychedelic agent is for administration at most once every twelve months.

The invention further provides, as embodiment 17, a combination, a method for treating, kit, dosage regime, method of adjuvant treatment, or short-duration psychedelic agent for use, according to any preceding embodiment, comprising
(i) an oral dosage form of the monoamine antidepressant agent; and
(ii) a parenteral dosage form of the short-duration psychedelic agent.

The invention further provides, as embodiment 18, a combination, a method for treating, kit, dosage regime, method of adjuvant treatment, or short-duration psychedelic agent for use, according to any preceding embodiment, for use in the treatment of a psychiatric disorder, preferably wherein the psychiatric disorder is selected from a depressive disorder, an anxiety disorder, obsessive-compulsive disorder, and an eating disorder, preferably selected from a depressive disorder and an anxiety disorder.

The invention further provides, as embodiment 19, a combination, a method for treating, kit, dosage regime, method of adjuvant treatment, or short-duration psychedelic agent for use, according to any preceding embodiment, for use in the treatment of a depressive disorder selected from major depressive disorder, persistent depressive disorder, bipolar disorder, bipolar depression, depression in terminally ill patients, and treatment resistant depression.

The invention further provides, as embodiment 20, a combination, a method for treating, kit, dosage regime, method of adjuvant treatment, or short-duration psychedelic agent for use, according to any preceding embodiment, for use in the treatment of an anxiety disorder selected from generalised anxiety disorder, phobia, panic disorder, social anxiety disorder, and post-traumatic stress disorder.

The invention further provides, as embodiment 21, a combination for use, a method for treating, kit, dosage regime, method of adjuvant treatment, or short-duration psychedelic agent for use, according to any one of embodiments 18 to 20, which further comprises the steps of and/or instructions for
assessing the patient with a baseline assessment using a rating scale and/or clinician reported outcomes and/or patient reported outcomes prior to administration of the short-duration psychedelic agent; and
assessing the patient with a post-dosing assessment following administration of the short-duration psychedelic agent.

The invention further provides, as embodiment 22, a combination for use, a method for treating, kit, dosage regime, method of adjuvant treatment, or short-duration psychedelic agent for use, according to embodiment 21, wherein the baseline assessment is by a rating scale selected from the Clinical Global Impression scale (CGI); the Montgomery-Asberg Depression Rating Scale (MADRS); Hamilton Depression Rating Scale (HAMD17); the Beck Depression Inventory-II; the Beck Anxiety Inventory (BAI); the Hamilton Anxiety Scale (HAM-A); the State-Trait Anxiety Inventory (STAI or STAI-T); Generalised Anxiety Disorder Assessment (GAD-7); the Clinician-Administered Posttraumatic Stress Disorder Scale (CAPS); the Yale-Brown Obsessive Compulsive Scale; the Beck Scale for Suicidal Ideation (BSS); Columbia-Suicide Severity Rating Scale (C-SSRS); the suicidal thoughts item of the MADRS for suicidal ideation; the Clinical Global Impression of Severity of Suicidality-Revised (CGI-SS-R) scale; the Beck Depression Inventory (BDI-II); the Quality of Life Enjoyment and Satisfaction Questionnaire-Short Form (Q-LES-Q-SF); the EQ-5D-5L descriptive system and EQ VAS; the Patient Global Impression of Severity scale (PGI-S); and combinations thereof.

The invention further provides, as embodiment 23, a combination for use, a method for treating, kit, dosage regime, method of adjuvant treatment, or short-duration psychedelic agent for use, according to embodiment 21 or 22, wherein the post-dosing assessment is by a rating scale selected from the Clinical Global Impression scale (CGI); the Montgomery-Asberg Depression Rating Scale (MADRS); the Hamilton Depression Rating Scale (HAMD17); the Beck Depression Inventory-II; the Beck Anxiety Inventory (BAI); the Hamilton Anxiety Scale (HAM-A); the State-Trait Anxiety Inventory (STAI or STAI-T); Generalised Anxiety Disorder Assessment (GAD-7); the Clinician-Administered Posttraumatic Stress Disorder Scale (CAPS); the Yale-Brown Obsessive Compulsive Scale; the Beck Scale for Suicidal Ideation (BSS); Columbia-Suicide Severity Rating Scale (C-SSRS); the suicidal thoughts item of the MADRS for suicidal ideation; the Clinical Global Impression of Severity of Suicidality-Revised (CGI-SS-R) scale; the Beck Depression Inventory (BDI-II); the Quality of Life Enjoyment and Satisfaction Questionnaire-Short Form (Q-LES-Q-SF); the EQ-5D-5L descriptive system and EQ VAS; the Patient Global Impression of Severity scale (PGI-S); the clinical global improvement scale (CGI-I); the Patient Global Impression of Improvement scale (PGI-I); and combinations thereof.

The invention further provides, as embodiment 24, a combination for use, a method for treating, kit, dosage regime, method of adjuvant treatment, or short-duration psychedelic agent for use, according to any one of embodiments 21 to 23, wherein the psychiatric disorder is a depressive disorder, and wherein the baseline and post-dosing assessment is carried out using a depression rating scale selected from the Montgomery-Asberg Depression Rating Scale (MADRS), Hamilton Depression Rating Scale (HAMD17), Beck Depression Inventory-II, and combinations thereof.

The invention further provides, as embodiment 25, a combination for use, a method for treating, kit, dosage regime, method of adjuvant treatment, or short-duration psychedelic agent for use, according to any one of embodiments 18 to 24, which provides a patient with remission from the psychiatric disorder.

The invention further provides, as embodiment 26, a combination for use, a method for treating, kit, dosage regime, method of adjuvant treatment, or short-duration psychedelic agent for use, according to embodiment 25, wherein remission is assessed at a time point from 6 to 10 days post administration with the short-duration psychedelic agent, preferably 6 to 8 days.

The invention further provides, as embodiment 27, a combination for use, a method for treating, kit, dosage regime, method of adjuvant treatment, or short-duration psychedelic agent for use, according to any preceding embodiment, wherein the administration or instructions for administration of the short-duration psychedelic agent comprises:
parenterally administering to the patient a dose of the short-duration psychedelic agent for a dosing period of up to about 15 minutes, preferably from about 5 to about 15 minutes, and wherein the parenteral administration is essentially continuous.

The invention further provides, as embodiment 28, a combination for use, a method for treating, kit, dosage regime, method of adjuvant treatment, or short-duration psychedelic agent for use, according to embodiments 11 to 27, wherein the administration of the short-duration psychedelic agent is parenteral administration selected from the group consisting of intravenous, intramuscular, intranasal, transmucosal, and transdermal administration and inhalation, preferably selected from intravenous and intramuscular administration.

The invention further provides, as embodiment 29, a combination for use, a method for treating, kit, dosage regime, method of adjuvant treatment, or short-duration psychedelic agent for use, according to embodiment 28, wherein the parenteral administration is by intravenous infusion, preferably wherein the intravenous administration is conducted using a syringe pump.

The invention further provides, as embodiment 30, a combination for use, a method for treating, kit, dosage regime, method of adjuvant treatment, or short-duration psychedelic agent for use, according to embodiment 29, wherein the parenteral administration is by a two-phase intravenous infusion.

The invention further provides, as embodiment 31, a combination for use, a method for treating, kit, dosage regime, method of adjuvant treatment, or short-duration psychedelic agent for use, according to embodiment 29, wherein the parenteral administration is by a single-phase intravenous infusion.

The invention further provides, as embodiment 32, a combination for use, a method for treating, kit, dosage regime, method of adjuvant treatment, or short-duration psychedelic agent for use, according to any one of embodiments 28 to 31, wherein the dosing period is from about 8 to about 12 minutes.

The invention further provides, as embodiment 33, a combination for use, a method for treating, kit, dosage regime, method of adjuvant treatment, or short-duration psychedelic agent for use, according to embodiment 30, wherein the dosing period is from about 9 to about 11 minutes, or about 10 minutes.

The invention further provides, as embodiment 34, a combination or combination for use, a method for treating, kit, dosage regime, method of adjuvant treatment, or short-duration psychedelic agent for use, according to any preceding embodiment, wherein the dose of the short-duration psychedelic agent is selected from the group consisting of:
about 20 to about 80 mg of N,N-dimethyltryptamine, or a pharmaceutically acceptable salt thereof,
about 20 to about 80 mg of deuterated N,N-dimethyltryptamine, or a pharmaceutically acceptable salt thereof;
about 15 to about 30 mg of deuterated N,N-dimethyltryptamine, or a pharmaceutically acceptable salt thereof;
about 10 to about 30 mg of 5-methoxy-N,N-dimethyltryptamine, a deuterated analogue, or a pharmaceutically acceptable salt thereof, and
about 10 to about 30 mg of deuterated 5-methoxy-N,N-dimethyltryptamine, or a pharmaceutically acceptable salt thereof;
   or the dose of the short-duration psychedelic agent is selected from the group consisting of:
about 10 to about 30 mg of psilocybin, or a pharmaceutically acceptable salt thereof,
about 10 to about 20 mg of deuterated psilocybin, or a pharmaceutically acceptable salt thereof,
about 1 to about 20 mg of psilocin, or a pharmaceutically acceptable salt thereof,
about 1 to about 20 mg of deuterated psilocin, or a pharmaceutically acceptable salt thereof, and
about 20 to about 50 mg of psilocin-4-glutarate, deuterated psilocin-4-glutarate, iprocin-4-glutarate or deuterated iprocin-4-glutarate, or a pharmaceutically acceptable salt thereof.

For the avoidance of doubt, when a salt of the short-duration psychedelic agent is used, the dose specified is the dose of the free-base equivalent.

The invention further provides, as embodiment 35, a combination for use according to any one of embodiments 11 to 34, or a method for treating, kit, dosage regime, method of adjuvant treatment, or short-duration psychedelic agent for use, according to any preceding embodiment, comprising the parenteral administration of a total dose of about 20 to about 29 mg of N,N-dimethyltryptamine, or a pharmaceutically acceptable salt thereof, or deuterated N,N-dimethyltryptamine, or a pharmaceutically acceptable salt thereof, or a mixture thereof.

The invention further provides, as embodiment 36, a combination for use according to any one of embodiments 11 to 35, or a method for treating, kit, dosage regime, method of adjuvant treatment, or short-duration psychedelic agent for use, according to any preceding embodiment, comprising the parenteral administration of a total dose of about 20 to about 23 mg, or a total dose of about 26 to about 29 mg of N,N-dimethyltryptamine, a deuterated analogue, a pharmaceutically acceptable salt thereof, or a mixture thereof.

The invention further provides, as embodiment 37, a combination for use according to any one of embodiments 11 to 34, or a method for treating, kit, dosage regime, method of adjuvant treatment, or short-duration psychedelic agent for use, according to any one of embodiments 2 to 34, wherein the short-duration psychedelic agent is N,N-dimethyltryptamine, or a pharmaceutically acceptable salt thereof, or deuterated N,N-dimethyltryptamine, or a pharmaceutically acceptable salt thereof, or a mixture thereof, and the total dose is about 7 to about 10 mg.

The invention further provides, as embodiment 38, a combination for use, a method for treating, kit, dosage regime, method of adjuvant treatment, or short-duration psychedelic agent for use, according to any one of embodiments 34 to 36, wherein the administration is by intravenous infusion, the short-duration psychedelic agent is N,N-dimethyltryptamine, or a pharmaceutically acceptable salt thereof, or deuterated N,N-dimethyltryptamine, a mixture thereof, or a pharmaceutically acceptable salt thereof, and the total dose is about 20 to about 23 mg.

The invention further provides, as embodiment 39, a combination for use, a method for treating, kit, dosage regime, method of adjuvant treatment, or short-duration psychedelic agent for use, according to any one of embodiments 34 to 36, wherein the administration is by intravenous infusion, the short-duration psychedelic agent is N,N-dimethyltryptamine, or a pharmaceutically acceptable salt thereof, or deuterated N,N-dimethyltryptamine, a mixture thereof or a pharmaceutically acceptable salt thereof, and the total dose is about 26 to about 29 mg.

The invention further provides, as embodiment 40, a combination for use, a method for treating, kit, dosage regime, method of adjuvant treatment, or short-duration psychedelic agent for use, according to embodiment 27, 28 and 34, wherein the administration is by intramuscular administration.

The invention further provides, as embodiment 41, a combination for use, a method for treating, kit, dosage regime, method of adjuvant treatment, or short-duration psychedelic agent for use, according to embodiment 40, wherein the short-duration psychedelic agent is N,N-dimethyltryptamine, or a pharmaceutically acceptable salt thereof, and the total dose is about 20 to about 70 mg, preferably about 50 to about 70 mg; or the short-duration psychedelic agent is N,N-dimethyltryptamine or deuterated N,N-dimethyltryptamine, or a mixture thereof, or a pharmaceutically acceptable salt thereof, and the total dose is about 20 to about 60 mg, or about 15 to about 30 mg.

The invention further provides, as embodiment 42, a combination, a combination for use, a method for treating, kit, dosage regime, method of adjuvant treatment, or short-duration psychedelic agent for use, according to any preceding embodiment, wherein the short-duration psychedelic agent is a compound of formula I, or a pharmaceutically acceptable salt thereof:

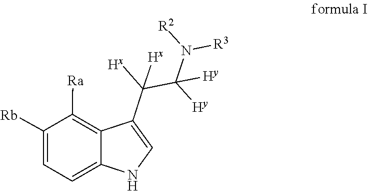

wherein:
Ra is selected from H, and D; or
Ra is selected from —OH, —OPO₃H₂ and —OC(O)C₁-C₃alkyleneCO₂H;
Rb is selected from H, D, —OCH₃ and —OCD₃;
R² and R³ are each independently selected from C(H^{z})₃; or
R² and R³ are each independently selected from C₂(H^{z})₅ and C₃(H^{z})₇; and each H^{x}, H^{y} and H^{z} is independently selected from protium and deuterium.

The invention further provides, as embodiment 43, a combination, a combination for use, a method for treating, kit, dosage regime, method of adjuvant treatment, or short-duration psychedelic agent for use, according to embodiment 42, wherein R² and R³ are each independently selected from CH₃ and CD₃.

The invention further provides, as embodiment 44, a combination, a combination for use, a method for treating, kit, dosage regime, method of adjuvant treatment, or short-duration psychedelic agent for use, according to embodiment 42, wherein R² and R³ are both CH₃, or R² and R³ are both CD₃, or R² is CH₃ and R³ is CD₃.

The invention further provides, as embodiment 45, a combination, a combination for use, a method for treating, kit, dosage regime, method of adjuvant treatment, or short-duration psychedelic agent for use, according to any one of embodiments 42 to 44, wherein each H^{x} is H, or each His D.

The invention further provides, as embodiment 46, a combination, a combination for use, a method for treating, kit, dosage regime, method of adjuvant treatment, or short-duration psychedelic agent for use, according to any one of embodiments 42 to 45, wherein each H^{y} is H, or each H^{y} is D, or one H^{y} is H and one H^{y} is D.

The invention further provides, as embodiment 47, a combination, a combination for use, a method for treating, kit, dosage regime, method of adjuvant treatment, or short-duration psychedelic agent for use, according to any one of embodiments 43 to 44 and 46, wherein each H^{x}, H^{y} and H^{z} is deuterium.

The invention further provides, as embodiment 48, a combination, a combination for use, a method for treating, kit, dosage regime, method of adjuvant treatment, or short-duration psychedelic agent for use, according to any preceding embodiment, wherein the short-duration psychedelic agent is selected from the group consisting of:

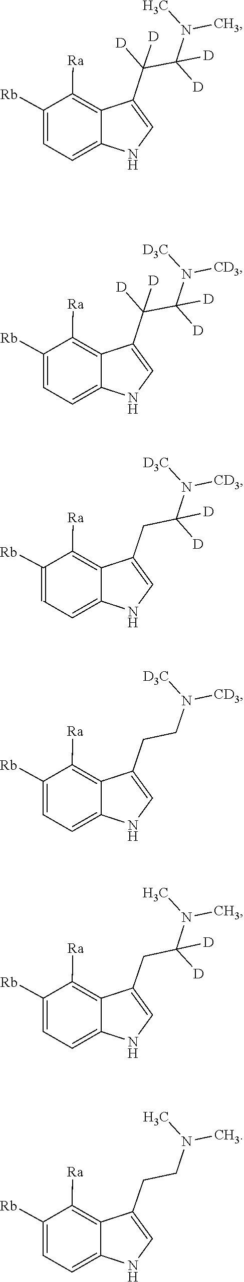

or a pharmaceutically acceptable salt thereof, or a mixture thereof, wherein Ra and Rb are as defined in embodiment 42.

The invention further provides, as embodiment 49, a combination, a combination for use, a method for treating, kit, dosage regime, method of adjuvant treatment, or short-duration psychedelic agent for use, according to any one of embodiments 42 to 48, wherein Ra is H.

The invention further provides, as embodiment 50, a combination, a combination for use, a method for treating, kit, dosage regime, method of adjuvant treatment, or short-duration psychedelic agent for use, according to any one of embodiments 42 to 49, wherein Rb is H.

The invention further provides, as embodiment 51, a combination, a combination for use, a method for treating, kit, dosage regime, method of adjuvant treatment, or short-duration psychedelic agent for use, according to any one of embodiments 42 to 50, wherein the compound is selected from:
N,N-dimethyltryptamine;
α-protio, α-deutero-N,N-dimethyltryptamine;
α,α-dideutero-N,N-dimethyltryptamine;
α,α,β,β-tetradeutero-N,N-dimethyltryptamine;
N,N-di(trideuteromethyl)tryptamine;
α-protio, α-deutero-N,N-di(trideuteromethyl)tryptamine;
α,α-dideutero-N,N-di(trideuteromethyl)tryptamine;
α,α,β,β-tetradeutero-N,N-di(trideuteromethyl)tryptamine;
5-methoxy-N,N-dimethyltryptamine;
5-methoxy-α-protio, α-deutero-N,N-dimethyltryptamine;
5-methoxy-α,α-dideutero-N,N-dimethyltryptamine;
5-methoxy-α,α,β,β-tetradeutero-N,N-dimethyltryptamine;
5-methoxy-N,N-di(trideuteromethyl)tryptamine;
5-methoxy-α-protio, α-deutero-N,N-di(trideuteromethyl)tryptamine;
5-methoxy-α,α-dideutero-N,N-di(trideuteromethyl)tryptamine;
5-methoxy-α,α,β,β-tetradeutero-N,N-di(trideuteromethyl)tryptamine; and

pharmaceutically acceptable salts thereof, and mixtures thereof, or the compound is selected from the group consisting of:
psilocybin;
α-protio, α-deutero-psilocybin;
α,α-dideutero-psilocybin;
α,α,β,β-tetradeutero-psilocybin;
4-(dihydrogen phosphate)-N,N-di(trideuteromethyl)tryptamine;
4-(dihydrogen phosphate)-α-protio, α-deutero-N,N-di(trideuteromethyl)tryptamine;
4-(dihydrogen phosphate)-α,α-dideutero-N,N-di(trideuteromethyl)tryptamine;
4-(dihydrogen phosphate)-α,α,β,β-tetradeutero-N,N-di(trideuteromethyl)tryptamine;
psilocin;
α-protio,α-deutero-psilocin;
α,α-dideutero-psilocin;
α,α,β,β-tetradeutero-psilocin;
4-hydroxy-N,N-di(trideuteromethyl)tryptamine;
4-hydroxy-α-protio,α-deutero-N,N-di(trideuteromethyl)tryptamine;
4-hydroxy-α,α-dideutero-N,N-di(trideuteromethyl)tryptamine;
4-hydroxy-α,α,β,β-tetradeutero-N,N-di(trideuteromethyl)tryptamine;
psilocin-4-glutarate;
α-protio,α-deutero-psilocin4-glutarate;
α,α-dideutero-psilocin-4-glutarate;
α,α,β,β-tetradeutero-psilocin-4-glutarate;
4-hydroxy-N,N-di(trideuteromethyl)tryptamine-4-glutarate;
4-hydroxy-α-protio,α-deutero-N,N-di(trideuteromethyl)tryptamine-4-glutarate;
4-hydroxy-α,α-dideutero-N,N-di(trideuteromethyl)tryptamine-4-glutarate;
4-hydroxy-α,α,β,β-tetradeutero-N,N-di(trideuteromethyl)tryptamine-4-glutarate;
iprocin-4-glutarate;
α-protio,α-deutero-iprocin4-glutarate;
α,α-dideutero-iprocin-4-glutarate;
α,α,β,β-tetradeutero-iprocin-4-glutarate;
4-hydroxy-N,N-di(heptadeuteroisopropyl)tryptamine-4-glutarate;
4-hydroxy-α-protio,α-deutero-N,N-di(heptadeuteroisopropyl)tryptamine-4-glutarate;
4-hydroxy-α,α-dideutero-N,N-di(heptadeuteroisopropyl)tryptamine-4-glutarate;
4-hydroxy-α,α,β,β-tetradeutero-N,N-di(heptadeuteroisopropyl)tryptamine-4-glutarate; and
   pharmaceutically acceptable salts thereof, and mixtures thereof.

The invention further provides, as embodiment 52, a combination for use, a method for treating, kit, dosage regime, method of adjuvant treatment, or short-duration psychedelic agent for use, according to any one of embodiments 18 to 51, comprising the administration to the patient, or instructions for the administration to the patient, of
(i) oral administration to the patient of a selective serotonin reuptake inhibitor over a period of 4 weeks or greater; and
(ii) parenteral administration to the patient of a short-duration psychedelic agent selected from N,N-dimethyltryptamine, 5-methoxy-N,N-dimethyltryptamine, deuterated analogues thereof, and pharmaceutically acceptable salts thereof.

Steps (i) and (ii) of embodiment 52 may be administered by different healthcare practitioners.

The invention further provides, as embodiment 53, a combination for use, a method for treating, kit, dosage regime, method of adjuvant treatment, or short-duration psychedelic agent for use, according to embodiment 52, wherein the oral administration to the patient of a selective serotonin reuptake inhibitor is over a period of 6 weeks or greater.

The invention further provides, as embodiment 54, a combination, a combination for use, a method for treating, kit, dosage regime, method of adjuvant treatment, or short-duration psychedelic agent for use, according to any preceding embodiment for use in the treatment of a psychiatric disorder in a patient, wherein the treatment comprises psychedelic-assisted psychotherapy.

The invention further provides, as embodiment 55, a kit according to claim 54, comprising instructions for psychedelic-assisted psychotherapy.

The invention further provides, as embodiment 56, a method for treating, dosage regime, combination for use, dosage regime, method of adjuvant treatment, or short-duration psychedelic agent for use, according to embodiment 54, comprising:
A. the administration to the patient of
(i) a monoamine antidepressant agent, and
(ii) a short-duration psychedelic agent, preferably a parenteral form of a short-duration psychedelic agent selected from N,N-dimethyltryptamine, deuterated N,N-dimethyltryptamine, 5-methoxy-N,N-dimethyltryptamine, deuterated 5-methoxy-N,N-dimethyltryptamine, psilocin, deuterated psilocin, psilocybin, deuterated psilocybin, psilocin-4-glutarate, deuterated psilocin-4-glutarate, iprocin-4-glutarate, deuterated iprocin-4-glutarate and pharmaceutically acceptable salts thereof, and
B. a psychotherapy session.

Steps (i) and (ii) of embodiment 56 may be administered by different healthcare practitioners.

The invention further provides, as embodiment 57, a method for treating, dosage regime, a combination for use, method of adjuvant treatment, or short-duration psychedelic agent for use, according to embodiment 54 or 56, wherein the psychotherapy session comprises:
a. Preparation Stage, comprising preparing the patient for a psychedelic experience;
b. Administration Stage, comprising the administration of the short-duration psychedelic agent according to any preceding embodiment; and
c. Integration Stage, comprising a psychiatrist or therapist led interview or discussion with the patient focused on the psychedelic experience.

The invention further provides, as embodiment 58, a kit according to embodiment 55, further comprising instructions for:
a. Preparation Stage, comprising preparing the patient for a psychedelic experience;
b. Administration Stage, comprising the administration of the short-duration psychedelic agent according to any preceding embodiment; and
c. Integration Stage, comprising a psychiatrist or therapist led interview or discussion with the patient focused on the psychedelic experience.

The invention further provides, as embodiment 59, a method of adjuvant treatment according to embodiment 5, wherein the short-duration psychedelic agent is administered to a patient who has received an adequate course of a monoamine antidepressant agent.

The invention further provides, as embodiment 60, a method of adjuvant treatment according to embodiment 5 or 59, wherein the short-duration psychedelic agent and the monoamine antidepressant agent are administered separately.

The invention further provides, as embodiment 61, a method of adjuvant treatment according to embodiment 5, 59, or 60 wherein
(i) the short-duration psychedelic agent is selected from N,N-dimethyltryptamine, deuterated N,N-dimethyltryptamine, and pharmaceutically acceptable salts thereof, and
(ii) the monoamine antidepressant agent is a selective serotonin reuptake inhibitor (SSRI) or serotonin-norepinephrine reuptake inhibitor (SNRI), preferably selected from citalopram, escitalopram, fluoxetine, fluvoxamine, paroxetine, sertraline, dapoxetine, and vortioxetine, and pharmaceutically acceptable salts thereof.

The invention further provides, as embodiment 62, a short-duration psychedelic agent for use according to embodiment 6, wherein the short-duration psychedelic agent is administered to a patient who has received an adequate course of a monoamine antidepressant agent.

The invention further provides, as embodiment 63, a short-duration psychedelic agent for use according to embodiment 6 or 62, wherein the short-duration psychedelic agent and the monoamine antidepressant agent are administered separately to a patient.

The invention further provides, as embodiment 64, a short-duration psychedelic agent for use according to embodiment 6, 62, or 63 wherein
(i) the short-duration psychedelic agent is selected from N,N-dimethyltryptamine, deuterated N,N-dimethyltryptamine, and pharmaceutically acceptable salts thereof, and
(ii) the monoamine antidepressant agent is a selective serotonin reuptake inhibitor (SSRI) or serotonin-norepinephrine reuptake inhibitor (SNRI), preferably selected from citalopram, escitalopram, fluoxetine, fluvoxamine, paroxetine, sertraline, dapoxetine, and vortioxetine, and pharmaceutically acceptable salts thereof.

The invention further provides, as embodiment 65, a combination comprising:
(i) a selective serotonin reuptake inhibitor (SSRI) or serotonin-norepinephrine reuptake inhibitor (SNRI), preferably selected from citalopram, escitalopram, fluoxetine, fluvoxamine, paroxetine, sertraline, dapoxetine, and vortioxetine, and pharmaceutically acceptable salts thereof; and
(ii) a short-duration psychedelic agent selected from N,N-dimethyltryptamine, deuterated N,N-dimethyltryptamine, and pharmaceutically acceptable salts thereof,
for the simultaneous, sequential or separate use in the treatment of a psychiatric disorder in a patient, preferably wherein the psychiatric disorder is selected from a depressive disorder, an anxiety disorder, obsessive compulsive disorder, and an eating disorder.

The invention further provides as embodiment 66, a combination for use according to embodiment 65, for the separate use in the treatment of a psychiatric disorder in a patient, preferably wherein the psychiatric disorder is selected from a depressive disorder, an anxiety disorder, obsessive compulsive disorder, and an eating disorder.

The invention further provides as embodiment 67, a combination for use according to embodiment 65 or 66, wherein the short-duration psychedelic agent is administered to a patient receiving who has received an adequate course of a selective serotonin reuptake inhibitor (SSRI) or serotonin-norepinephrine reuptake inhibitor (SNRI).

The invention further provides, as embodiment 68, a combination for use according to embodiment 65 to 67 comprising an SSRI selected from citalopram, escitalopram, fluoxetine, fluvoxamine, paroxetine, sertraline, dapoxetine, and vortioxetine, and pharmaceutically acceptable salts thereof.

The invention further provides, as the seventh aspect, the use of a short-duration psychedelic agent for the manufacture of a medicament for the adjuvant treatment of a psychiatric disorder in a patient receiving treatment with a monoamine antidepressant agent, preferably wherein the psychiatric disorder is selected from a depressive disorder, an anxiety disorder, obsessive compulsive disorder, and an eating disorder.

The invention provides, as the eighth aspect, a parenteral formulation comprising a dose of a short-duration psychedelic agent for use in the treatment of a psychiatric disorder in a patient receiving treatment with a monoamine antidepressant agent; wherein the treatment comprises the administration, preferably parenteral administration, to the patient of the dose of the short-duration psychedelic agent; preferably wherein the psychiatric disorder is selected from a depressive disorder, an anxiety disorder, obsessive-compulsive disorder, and an eating disorder, preferably selected from a depressive disorder and an anxiety disorder. The depressive disorder may be selected from major depressive disorder, persistent depressive disorder, bipolar disorder, bipolar depression, depression in terminally ill patients, and treatment resistant depression. The anxiety disorder may be selected from generalised anxiety disorder, phobia, panic disorder, social anxiety disorder, and post-traumatic stress disorder.

The invention further provides, as the ninth aspect, a delivery device for use in the treatment of a psychiatric disorder in a patient receiving treatment with a monoamine antidepressant agent, wherein the delivery device is configured to deliver the administration, preferably parenteral administration, to the patient of a parenteral dose of a short-duration psychedelic agent.

For the avoidance of doubt, all embodiments and aspects disclosed herein in relation to the first to sixth aspects apply mutatis mutandis to the seventh to ninth aspect of the invention.

The combination of the monoamine antidepressant agent and the short-duration psychedelic agent, as defined in any aspect or embodiment of the present invention, when administered to a patient, preferably provides simultaneous detectable plasma levels of the monoamine antidepressant agent and the short-duration psychedelic agent.

The short-duration psychedelic agents for use in the combination, combination for use, dosage regimen, method for treating, method of adjuvant treatment, short-duration psychedelic agent for use, kit, delivery device, or parenteral formulation for use of the invention may be prepared according to the synthetic processes described in WO2021/089873, US20210395201, WO2022/117359 and US20220202775, the disclosures of which are hereby incorporated by reference in their entireties.

The dose of short-duration psychedelic agent for use in the combination, combination for use, dosage regimen, method for treating, method of adjuvant treatment, short-duration psychedelic agent for use, kit, delivery device, or parenteral formulation for use of the invention may preferably be in the form of a pharmaceutically acceptable salt, wherein the salt comprises an acid and the freebase of a short-duration psychedelic agent selected from N,N-dimethyltryptamine, 5-methoxy-N,N-dimethyltryptamine, psilocybin, psilocin, psilocin-4-glutarate, iprocin-4-glutarate, and deuterated analogues thereof. An example of a salt comprising an acid and dimethyltryptamine compound is N,N-dimethyltryptamine fumarate, which is the fumaric acid salt of N,N-dimethyltryptamine. P. H. Stahl and C. G. Wermuth provide an overview of pharmaceutical salts and the acids comprised therein in Handbook of Pharmaceutical Salts: Properties, Selection and Use, Weinheim/Zurich:Wiley-VCH/VHCA, 2002. The acids described in this review are suitable acids for inclusion within the salt of the formulation.

The salt may comprise an acid selected from the group consisting of fumaric acid, tartaric acid, citric acid, acetic acid, lactic acid, gluconic acid, 1-hydroxy-2-naphthoic acid, 2,2-dichloroacetic acid, 2-hydroxyethanesulfonic acid, 2-oxoglutaric acid, 4-acetamidobenzoic acid, 4-aminosalicylic acid, adipic acid, ascorbic acid, aspartic acid, benzenesulfonic acid, benzoic acid, camphoric acid, camphor-10-sulfonic acid, decanoic acid, hexanoic acid, octanoic acid, carbonic acid, cinnamic acid, cyclamic acid, dodecylsulfuric acid, ethane-1,2-disulfonic acid, ethanesulfonic acid, formic acid, galactaric acid, gentisic acid, glucoheptonic acid, glucuronic acid, glutamic acid, glutaric acid, glycerophosphoric acid, glycolic acid, hippuric acid, hydrobromic acid, hydrochloric acid, isobutyric acid, lactobionic acid, lauric acid, maleic acid, malic acid, malonic acid, mandelic acid, methanesulfonic acid, naphthalene-1,5-disulfonic acid, naphthalene-2-sulfonic acid, nicotinic acid, nitric acid, oleic acid, oxalic acid, palmitic acid, pamoic acid, phosphoric acid, proprionic acid, pyroglutamic acid (-L), salicylic acid, sebacic acid, stearic acid, succinic acid, sulfuric acid, thiocyanic acid, toluenesulfonic acid and undecylenic acid.

Preferably, the pharmaceutically acceptable salt is selected from fumarate, tartarate, citrate, succinate, benzoate, and hydrochloride. More preferably, the pharmaceutically acceptable salt is fumarate.

More preferably, the pharmaceutically acceptable salt of the short-duration psychedelic agent is the fumarate salt.

Parenteral routes of administration include intravenous, intramuscular, subcutaneous, intranasal, transmucosal, sublingual, rectal, and transdermal administration, and inhalation. Any parenteral routes capable of administration over a period of up to about 15 minutes, preferably for a period of about 5 to about 15 minutes, are suitable for use in the present invention. Preferred routes of parenteral administration according to any aspect of the present invention are selected from intravenous infusion, intramuscular infusion, subcutaneous infusion, intranasal, transmucosal, and transdermal administration, and inhalation. Intravenous infusion is a particularly preferred route of administration. Intramuscular administration is a particularly preferred route of administration.

When the parenteral route of administration is intravenous, the delivery device may comprise an infusion bag or a syringe, and may preferably further comprise a syringe pump. Where two syringe pumps are used to administer the intravenous infusion, the syringe pumps may be joined via a 3-way tap into a single cannula. When the parenteral route of administration is intramuscular, the delivery device may comprise a syringe. When the parenteral route is intranasal, the delivery device may comprise a pump-action spray means. When the parenteral route is transmucosal, the delivery device may comprise an oromucosal film. When the parenteral route is transdermal, the delivery device may comprise a transdermal patch. When the route of administration is inhalation, the delivery device may comprise a metered dose inhaler, or a vaporiser, or a nebuliser.

Dosage forms suitable for parenteral administration have a pH of about 3 to 9 and, for liquid formulations, an osmolality of about 250 to about 600 mOsm/Kg. pH values above 9 are reported by I. Usach et al. in Adv. Ther., 36, 2986-2996 (2019) to relate to tissue necrosis (death of cells within the tissue), whereas values lower than 3 are reported to cause pain and phlebitis (inflammation of veins). Osmolality values greater than 600 mOsm/Kg are also reported to cause pain. Usach et al also recommend that parenteral formulations should be formulated as isotonic solutions (osmolality of about 300 mOsm/Kg), proposing an upper limit of 600 mOsm/Kg to minimise pain.

Osmolality is formally defined as the quotient of the negative natural logarithm of the rational activity of water and the molar mass of water, as represented by formula:

$$osmolality = \frac{- ln {a}_{w}}{18.015} ; {a}_{w} = \frac{p}{{p}^{*}}$$

where p is the partial vapour pressure of water in the solution and p* is the partial vapour pressure of pure water. In simpler terms, osmolality is the number of osmotically active particles (the number of solute particles) in 1 kg of a solution. Thus, osmolality is a function only of the number of particles, and is not related to particle molecular weight, size, shape, or charge (see D. K. Faria et al., M. E. Mendes and N. M. Sumita, J. Bras. Patol. Med. Lab., 53, 1, 38-45 (2017) for a review of the measurement of serum osmolality). For example, one mole of a nondissociating substance (e.g., DMT as a free base) dissolved in 1 kg of water has an osmolality of 1 Osm/kg (1000 mOsm/kg), whilst one mole of a substance that dissociates into two separate species in solution (e.g., DMT fumarate) dissolved in 1 kg of water has an osmolality of 2 Osm/kg (2000 mOsm/kg).

Where a first solution is defined herein to be isotonic with a second solution, the solutions have the same osmolality. For example, where a formulation is defined to be isotonic with human blood serum, the formulation has the same osmolality as human blood serum. Human blood serum typically has an osmolality of about 275 to about 300 mOsm/Kg (L. Hooper et al., BMJ Open, 2015; 5(10): e008846).

Suitable formulations for injection according to the present invention are described in WO2022/043227 and U.S. Ser. No. 11/406,619. Suitable formulations for administration by inhalation in accordance with the invention are described in WO2022/117640. Suitable formulations for transdermal administration are described in US 20210346347 and U.S. Ser. No. 17/866,477. Suitable formulations for buccal administration are described in WO2022/232179. Other suitable formulations according to the present invention are described in co-pending patent application U.S. Ser. No. 17/574,424. The entire disclosures of each of these patent applications are hereby incorporated by reference in their entireties.

In some embodiments, the parenteral formulation for use in the present invention comprises a salt of the short-duration psychedelic agent, a base agent, water, and optionally a buffer which is separate to the salt, wherein the formulation has a pH of from about 5 to about 6, a concentration of about 10 mg/ml as freebase or greater, and an osmolality of from about 250 to about 350 mOsm/Kg; and wherein the formulation comprises a dose of the short-duration psychedelic agent within a volume of 5 ml or less.

The base agent adjusts the pH of the formulation to the required pH range, for example from pH 5 to pH 6. The pH of a formulation including the short-duration psychedelic agent salt, water, and a buffer is often low, e.g., less than pH 5, and so a pH adjustment with a base agent may be required. The skilled person is able to assess suitable base agents to adjust the pH of the solution without risk of degradation of the short-duration psychedelic agent salt. The base agent may be sodium hydroxide or potassium hydroxide.

The parenteral formulation optionally comprises a buffer, which is separate to the salt, i.e., the buffer is not merely a counterion to the short-duration psychedelic agent. For example, where the salt is N,N-dimethyltryptamine fumarate (i.e., the fumaric acid salt of N,N-dimethyltryptamine), an amount of buffer may be required over and above the buffer effect provided by the fumarate salt. The term ‘buffer’ is well known in the art and refers to a chemical which, on inclusion within a formulation, resists a change in pH on addition of acid or base to the formulation. As used herein, the term ‘buffer’ refers to the buffer system or the buffer agent. Within a formulation, a buffer comprises a weak acid and its conjugate base. A suitable buffer comprises an acid with a pKa value that lies within f1 of the desired pH of the formulation. For example, if the desired pH of the formulation is about 5.0, a suitable buffer comprises a weak acid with a pKa value of from about 4.0 to about 6.0. If the acid of a buffer has more than one pKa value (i.e., each molecule of the acid is able to donate more than one proton), in order for the buffer to be suitable, at least one of the pKa values lies within the desired pH range.

The weak acid and conjugate base of the buffer are in equilibrium with one another. In accordance with Le Chatelier's principle (if a constraint (such as a change in concentration of a reactant) is applied to a system in equilibrium, the equilibrium will shift so as to counteract the effect of the constraint), addition of acid or base to the formulation shifts the position of equilibrium in favour of the conjugate base or weak acid, respectively. Consequently, the concentration of free protons in the formulation (and thus the pH) is relatively unchanged.

Suitable buffer systems comprise an acetate salt and acetic acid (pKa=4.75); a citrate salt and citric acid (pKa=3.13, 4.76 and 6.40); and phosphoric acid (pKa=2.14, 7.20 and 12.37); or mixtures thereof. The pKa values cited herein are those reported at 25° C. in water. Typically, the buffer comprises only one of the pairs listed above, i.e., one acid and its conjugate base.

In some embodiments, the buffer comprises an acetate salt and acetic acid; a citrate salt and citric acid; or a phosphate salt and phosphoric acid. Sometimes, the buffer comprises an acetate salt and acetic acid; or a citrate salt and citric acid. In some embodiments, the buffer comprises an acetate salt and acetic acid, often sodium acetate and acetic acid, or potassium acetate and acetic acid.

The concentration of buffer within the formulation is typically sufficient to resist significant pH change of the formulation on storage of the formulation for two weeks (i.e., the pH typically fluctuates less than about 0.1 pH unit), The skilled person is able to assess suitable buffer concentrations and to achieve this. Often, the concentration of buffer is from about 15 mM to about 75 mM, such as about 20 mM to about 30 mM. In some embodiments, the concentration of the buffer is about 25 mM.

As used herein, the term ‘buffer agent’ refers to the weak acid or weak base. Any pharmaceutically acceptable buffer agent may be used in the formulations of the invention, including phosphoric acid, citric acid, acetic acid, phosphate salt, citrate salt, and acetate salt. In some embodiments, the buffer comprises sodium phosphate, sodium citrate, or sodium acetate.

Sometimes, the concentration of the short-duration psychedelic agent and optional buffer in the formulation gives rise to the desired osmolality. Alternatively, the desired osmolality may be achieved by inclusion of one or more tonicity agents in the formulation. Thus, in some embodiments, the formulation further comprises a tonicity agent. A tonicity agent is defined herein as a chemical that, on inclusion within a formulation, increases the osmolality of the formulation. As described above, the osmolality is the number of osmotically active particles (the number of solute particles) in 1 kg of a solution. Thus, a chemical that acts as a solute when incorporated into the formulation lies within the definition of a tonicity agent.

In some embodiments, the formulation comprises a tonicity agent. The concentration of tonicity agent depends on the concentration of other components within the formulation, such as the short-duration psychedelic agent and buffer. For example, where the formulation without tonicity agent has an osmolality of about 60 mOsm/kg, at least about 190 mOsm/kg would be provided by a tonicity agent (e.g., 95 mM of sodium chloride). M. F. Powell, T. Nguyen and L. Baloian provide a review of excipients suitable for parenteral administration (administration other than by the mouth or alimentary canal) in PDA J. Pharm. Sci. Technol., 52, 238-311 (1998). All soluble excipients listed in this review article that can be given by the intravenous route will, when added to the formulation, contribute to the osmolality and thus can be considered tonicity agents.

Suitable excipients for use in the dose of a short-duration psychedelic agent when used in accordance with the invention may be selected from the group comprising ethanol, citric acid, trisodium citrate, benzalkonium chloride, microcrystalline cellulose, carboxymethylcellulose sodium, chlorobutanol, edetate disodium, glycerin, hydrochloric acid, methylparaben, polyethylene glycol, propylene glycol, propylparaben, saccharin sodium, sodium bicarbonate, sodium bisulphate, sodium bisulphite, sodium chloride, sodium hydroxide, sodium metabisulphite, sodium phosphate, sodium citrate, sulphuric acid, trisodium citrate, tromethamine, and mixtures thereof.

Some excipients may act as a cosolvent. Suitable solvents or cosolvents for use in the formulations of the invention may be selected from ethanol, polyethylene glycol, propylene glycol, and mixtures thereof. In some embodiments, the formulation comprises a cosolvent. In some embodiments, the formulation does not comprise a cosolvent. In particular, when the salt of the short-duration psychedelic agent is a fumarate, for example N,N-dimethyltryptamine fumarate or α,α-dideutero-N,N-dimethyltryptamine fumarate, the formulation does not comprise a cosolvent.

Therapeutic improvement may be assessed using a rating scale commonly used in the field. For patients with a depressive disorder such as MDD or TRD, the Montgomery-Asberg Depression Rating Scale (MADRS) or the Hamilton Depression Rating Scale (HAMD-17) may be used. The rating scale is used prior to administration of the short-duration psychedelic agent, to provide a baseline assessment, and again after administration of the short-duration psychedelic agent (post-dosing) to assess the therapeutic effect. Preferably post-dosing assessment occurs from about 24 hours to about one month after administration of the short-duration psychedelic agent, preferably about one week, about two weeks, about three weeks and/or about four weeks after administration of the short-duration psychedelic agent. A reduction in symptoms to an essentially asymptomatic level is considered to indicated remission. Remission may be assessed by a MADRS score of 13 or below, or 10 or below, a HAMD-17 score of 10 or below, preferably 8 or below, or a BDI-II score of 13 or below, preferably 11 or below.

In some embodiments, the short-duration psychedelic agent may be administered as a two-phase IV infusion through a venous cannula over 6-11 min. In some embodiments, the short-duration psychedelic agent may be administered as a two-phase IV infusion through a venous cannula, wherein each phase comprises a 5-minute infusion. In some embodiments, the short-duration psychedelic agent may be administered as a single-phase IV infusion through a venous cannula over 6-11 min, preferably about 10 minutes. Where a two-phase IV infusion is used, suitably the syringe pumps are joined via a 3-way tap into a single cannula: once infusion is complete on the first pump, the 3-way tap is turned to allow infusion to continue from the second pump.

The term ‘single-phase’ IV infusion refers to the administration of an IV infusion by one syringe pump, with the administration of the short-duration psychedelic agent from a single syringe pump. The term ‘two-phase’ IV infusion refers to the administration of an IV infusion by two syringe pumps, with the first phase comprising administration of the short-duration psychedelic agent from the first syringe pump, followed by the second phase comprising administration of the short-duration psychedelic agent from the second syringe pump. The two-phase IV infusion is essentially continuous, with a non-material break in the infusion to change administration from the first syringe pump to the second syringe pump.

In some embodiments, the short-duration psychedelic agent may be administered by intramuscular administration.

In some embodiments, the kit is a kit for treating a psychiatric disorder in a patient, according to any preceding embodiment, and the kit includes a) a container comprising a parenteral dose of a short-duration psychedelic agent selected from N,N-dimethyltryptamine, deuterated N,N-dimethyltryptamine, 5-methoxy-N,N-dimethyltryptamine, deuterated 5-methoxy-N,N-dimethyltryptamine, and pharmaceutically acceptable salts thereof, and b) a label or package insert on or associated with the container comprising instructions for administration of the parenteral formulation, indicating that the administration to a patient is in conjunction with the administration of monoamine antidepressant, preferably a selective serotonin reuptake inhibitor or serotonin-norepinephrine reuptake inhibitor.

In some embodiments, the parenteral formulation, combination, combination for use, method of treating, method of adjuvant treatment, short-duration psychedelic agent for use, kit, delivery device, or dosage regime according to any preceding embodiment is for use in the treatment of a patient who has not responded to an adequate course of treatment with at least one antidepressant agent, preferably at least one monoamine antidepressant, or at least two different antidepressant agents.

The term ‘not responded’ as used herein refers to a lack of clinically meaningful improvement in symptoms, for example as measured by one or more of the rating scales described hereinabove. The lack of response to an adequate course of treatment with a monoamine antidepressant agent may be determined retrospectively or prospectively. Prospective determination of lack of response refers to a determination made by the prescribing clinician or therapist following administration of part of a course of treatment. Retrospective determination refers to a determination made by the prescribing clinician or therapist following administration of a full adequate course of treatment.

The present invention is directed to treatment of a disorder with a combination of agents; a monoamine antidepressant agent, and a short-duration psychedelic agent. A ‘therapeutically effective’ dose of the two agents refers to an amount of the monoamine antidepressant agent, and the short-duration psychedelic agent, which when administered in combination, elicits a clinically meaningful response in a patient. The dose of each agent, when administered as a single agent may not be therapeutically effective. For example, the dose of the monoamine antidepressant agent may be less than a therapeutically effective amount when administered as a single agent, but may be therapeutically effective when administered in combination with the short-duration psychedelic agent in accordance with the present invention. For the avoidance of doubt, the dose of the (i) monoamine antidepressant agent, and (ii) the short-duration psychedelic agent, as defined in any preceding embodiment, comprise in combination a therapeutically effective dose.

Combination treatment according to the present invention can be achieved by administering a monoamine antidepressant agent and a short-duration psychedelic agent, simultaneously, sequentially, or separately. The monoamine antidepressant agent and the short-duration psychedelic agent are preferably formulated and administered separately. The monoamine antidepressant agent and the short-duration psychedelic agent are often prescribed and/or administered by different healthcare practitioners (such as physicians). The invention also provides for administering the two in a single formulation. Other combinations may also be encompassed by combination therapy. For example, two agents can be formulated together and administered in conjunction with a separate formulation containing a further dose of one of the agents, or a third agent. While the two or more agents in the combination treatment can be administered simultaneously, they need not be. For example, administration of a first agent (or combination of agents) can precede administration of a second agent (or combination of agents) by minutes, hours, days, or weeks. Thus, the two or more agents can be administered within minutes of each other or within 1, 2, 3, 6, 9, 12, 15, 18, or 24 hours of each other or within 1, 2, 3, 4, 5, 6, 7, 8, 9, 10, 12, 14 days of each other or within 2, 3, 4, 5, 6, 7, 8, 9, or 10 weeks of each other, for example where the monoamine antidepressant is administered as a controlled release dosage form. In some cases, even longer intervals are possible. While in many cases it is desirable that the two or more agents used in a combination treatment be present within the patient's body at the same time, that is, the administration regimes of the monoamine antidepressant and the short-duration psychedelic agent provide detectable plasma levels at the same time, this need not be so. Combination treatment can also include two or more administrations of one or more of the agents used in the combination. The two agents may be administered according to different dosing schedules, and by different healthcare practitioners. For example, the monoamine antidepressant agent may be delivered on a daily schedule, with separate administration of a single dose of the short-duration psychedelic agent in any three-month period, or six-month, or nine-month, or twelve-month period. For example, the monoamine antidepressant agent may be delivered on a daily schedule, with separate administration of a single dose of the short-duration psychedelic agent at most once every three months, or at most once every six months, or at most once every nine months, or at most once every twelve months. The two agents of the present invention may be administered sequentially in any combination one or more times. Preferably, the combination treatment according to the present invention is achieved by administration regimes of the antidepressant agent and the short-duration psychedelic agent which provide simultaneous detectable plasma levels of the two agents at the same time.

### EXAMPLES

An unblinded study of a single dose of N,N-dimethyltryptamine [DMT] fumarate in patients currently taking a selective serotonin reuptake inhibitor (SSRI) for their depression, but for whom the SSRI is not fully relieving their depression is ongoing (ClinicalTrials.gov Identifier: NCT05553691). The study comprises two groups:

“Test Cohort”: Patients who are currently on a stable treatment course of SSRIs, which have been ineffective in fully relieving their depression symptoms (SSRI prescribed outside of study). Patients were administered a single dose of DMT fumarate by intravenous infusion; and

“Control Cohort”: Patients who are not currently taking any pharmacological treatment for their depression (control group). Patients were administered a single dose of DMT fumarate by intravenous infusion.

Patients were recruited with moderate-severe Major Depressive Disorder (“MDD”) as defined by a Hamilton Depression Rating Scale (HAM-D) score of ≥14. Efficacy was assessed using the Montgomery-Asberg Depression Rating scale (MADRS) to measure any change in patients' depression symptoms from baseline (Day 0). Additional exploratory endpoints include the Beck Depression Inventory (BDI) to assess patients' self-reported depression, and the State-Trait Anxiety Inventory (STAI-T) to assess patients' self-reported anxiety.

DMT fumarate is administered by intravenous infusion on Day 1.

### Efficacy

### MADRS

Results on the MADRS scale are shown in Table 1 below.

TABLE 1
MADRS

| Test Cohort | | | | | Control Cohort | | | | |
|---|---|---|---|---|---|---|---|---|---|
| Day | 0 | 8 | 15 | 29 | Day | 0 | 8 | 15 | 29 |
| S1001 | 28 | 2 | 4 | 1 | S2001 | 37 | 21 | 18 | 18 |
| S1003 | 36 | 1 | 9 | 0 | S2002 | 35 | 21 | 21 | 15 |
| S1004 | 25 | 8 | 0 | 2 | S2003 | 33 | 24 | 26 | 30 |
| S1005 | 31 | 2 | 0 | 0 | S2004 | 33 | 9 | 7 | 0 |
| S1006 | 13 | 1 | 1 | 0 | S2005 | 39 | 7 | 8 | 17 |
| S1007 | 29 | 0 | 0 | 0 | | | | | |
| S1008 | 36 | 27 | 27 | 8 | | | | | |
| S1009 | 28 | 21 | 15 | 11 | | | | | |
| S1010 | 23 | 0 | 0 | 1 | | | | | |
| S1011 | 32 | 10 | 8 | 6 | | | | | |
| S1012 | 33 | 1 | 3 | 0 | | | | | |
| S1013 | 32 | 7 | 5 | 7 | | | | | |
| n | 12 | 12 | 12 | 12 | n | 5 | 5 | 5 | 5 |
| Mean | 28.8 | 6.7 | 6.0 | 3.0 | Mean | 35.4 | 16.4 | 16.0 | 16.0 |
| SD | 6.4 | 8.8 | 8.1 | 3.9 | SD | 2.6 | 7.8 | 8.3 | 10.7 |
| Mean % | | −78.1 | −81.2 | −90.2 | Mean % | | −53.1 | −54.2 | −54.8 |
| change from | | | | | change from | | | | |
| Baseline | | | | | Baseline | | | | |
| Response* | | | | 100% | Response* | | | | 80% |
| Remission* | | | | 92% | Remission* | | | | 20% |

*Response is defined as ≥50% reduction in MADRS score from baseline; Remission is defined as a MADRS score ≤10.

It can be seen that treatment with DMT fumarate in combination with an SSRI (test cohort) resulted in a mean % change from baseline (Day 0) of from −78.1% at day 8, to −90.2% at day 29 for participants in the test cohort (n=12). The response rate was 100% at day 29 for the test cohort. It is surprising that at day 29 complete remission was observed in 11 participants in this test cohort (92%), while 10 participants exhibited remission at all other time points. One participant was observed to have a MADRS score of 11 at day 29, just missing the criteria for remission at 10.

In comparison, the control cohort (n=5) exhibited a mean % change from baseline of −53.1% on day 8 and −54.8% on day 29, with a response rate at day 29 of 80%. One participant in the control cohort exhibited remission at Day 29 (20%).

It was surprising to observe such a marked difference in efficacy between the antidepressant effect of the DMT treatment in the test cohort compared to the control cohort. These data suggest a potentially enhanced efficacy effect when DMT fumarate is administered in combination with SSRIs.

### BDI-II

These data were corroborated by assessment on the Becks (BDI-II) scale at day 15 and day 29, as shown in Table 2 below:

TABLE 2
BDI-II

| Test Cohort | | | | Control Cohort | | | |
|---|---|---|---|---|---|---|---|
| Day | 0 | 15 | 29 | Day | 0 | 15 | 29 |
| S1001 | 33 | 0 | 0 | S2001 | 42 | 17 | 13 |
| S1003 | 46 | 5 | 0 | S2002 | 37 | 17 | 16 |
| S1004 | 25 | 1 | 0 | S2003 | 33 | 22 | 31 |
| S1005 | 40 | 0 | 0 | S2004 | 27 | 8 | 2 |
| S1006 | 18 | 0 | 0 | S2005 | 41 | 20 | 12 |
| S1007 | 28 | 3 | 4 | | | | |
| S1008 | 41 | 29 | 11 | | | | |
| S1009 | 14 | 6 | 7 | | | | |
| S1010 | 21 | 3 | 1 | | | | |
| S1011 | 33 | 5 | 7 | | | | |
| S1012 | 28 | 1 | 2 | | | | |
| S1013 | 33 | 8 | 5 | | | | |
| n | 12 | 12 | 12 | n | 5 | 5 | 5 |
| Mean | 30.0 | 5.1 | 3.1 | Mean | 36.0 | 16.8 | 14.8 |
| SD | 6.6 | 1.9 | 1.0 | SD | 7.6 | 3.7 | 3.7 |
| Mean % | | −84 | −88 | Mean % | | −54 | −59 |
| change from | | | | change from | | | |
| Baseline | | | | Baseline | | | |

Again, treatment with DMT fumarate resulted a significant mean % change from baseline (Day 0) of from −84% at day 15, to −88% at day 29 for participants in the test cohort (n=12). This compares with a mean % change from baseline of from −54% at day 15, to −59% at day 29 for participants in the control cohort (n=5).

### STAI-T

Results on the STAI-T scale are shown below.

TABLE 3
STAI-T

| Test Cohort | | | | | Control Cohort | | | | |
|---|---|---|---|---|---|---|---|---|---|
| Day | 0 | 8 | 15 | 29 | Day | 0 | 8 | 15 | 29 |
| S1001 | 71 | 34 | 33 | 34 | S2001 | 72 | 40 | 46 | 49 |
| S1003 | 69 | 28 | 25 | 26 | S2002 | 69 | 44 | 39 | 47 |
| S1004 | 56 | 29 | 22 | 23 | S2003 | 59 | 50 | 52 | 59 |
| S1005 | 61 | 38 | 32 | 32 | S2004 | 58 | 38 | 41 | 32 |
| S1006 | 50 | 47 | 44 | 45 | S2005 | 70 | 60 | 58 | 51 |
| S1007 | 63 | 40 | 30 | 34 | | | | | |
| S1008 | 67 | 66 | 62 | 41 | | | | | |
| S1009 | 48 | 49 | 45 | 43 | | | | | |
| S1010 | 59 | 34 | 33 | 28 | | | | | |
| S1011 | 70 | 40 | 40 | 40 | | | | | |
| S1012 | 56 | 26 | 42 | 32 | | | | | |
| S1013 | 61 | 46 | 40 | 44 | | | | | |
| n | 12 | 12 | 12 | 12 | n | 5 | 5 | 5 | 5.0 |
| Mean | 60.9 | 39.8 | 37.3 | 35.2 | Mean | 65.6 | 46.4 | 47.2 | 47.6 |
| SD | 7.6 | 11.2 | 10.7 | 7.4 | SD | 5.9 | 7.9 | 7.0 | 8.8 |
| Mean % | — | −33.6 | −37.5 | −41.0 | Mean % | — | −28.9 | −27.6 | −27.2 |
| change from | | | | | change from | | | | |
| Baseline | | | | | Baseline | | | | |

It can be seen from the data presented in Table 3 that treatment with DMT fumarate resulted a significant mean % change from baseline (Day 0) of −41.0% at day 29 for participants in the test cohort (n=12). This compares with a mean % change from baseline of −27.2% at day 29 for participants in the control cohort (n=5).

The data presented above in Tables 1 to 3 show a significant reduction in symptoms of both depression and anxiety following treatment with DMT fumarate for patients taking a selective serotonin reuptake inhibitor (SSRI) for their depression, but for whom the SSRI is not fully relieving their depression.

### Safety & Tolerability

The administration of DMT fumarate was well-tolerated by all patients in both the test and the control cohorts, with no apparent differences between cohorts. No drug-related serious adverse events reported. A small number of drug-related adverse events (“AEs”) were reported (8 in the test cohort and 3 in the control cohort), all of which were deemed to be mild or moderate in severity. The majority of drug-related AEs were resolved during the dosing visit.

1. A method for treating a psychiatric disorder in a patient, wherein the psychiatric disorder is at least one selected from a depressive disorder, an anxiety disorder, obsessive compulsive disorder, and an eating disorder, the method for treating comprising administering to the patient:
(i) a monoamine antidepressant agent selected from a selective serotonin reuptake inhibitor and a serotonin-norepinephrine reuptake inhibitor; and
(ii) a short-duration psychedelic agent selected from N,N-dimethyltryptamine and pharmaceutically acceptable salts thereof, deuterated N,N-dimethyltryptamine and pharmaceutically acceptable salts thereof, and mixtures thereof.

2. The method for treating according to claim 1, wherein the administration of
(i) the monoamine antidepressant agent; and
(ii) the short-duration psychedelic agent
provides a synergistic effect.

3. The method for treating according to claim 1, wherein the monoamine antidepressant agent is at least one selected from desvenlafaxine, duloxetine, levomilnacipran, milnacipram, venlafaxine, citalopram, escitalopram, fluoxetine, fluvoxamine, paroxetine, sertraline, dapoxetine, and vortioxetine, and the pharmaceutically acceptable salts thereof.

4. The method for treating according to claim 1, comprising:
(i) administration of an adequate course of the monoamine antidepressant agent; and
(ii) administration of a single dose of the short-duration psychedelic agent.

5. A method for treating according to claim 4, wherein the adequate course of the monoamine antidepressant agent comprises a period of treatment of 4 weeks or more.

6. The method for treating according to claim 1, comprising
(i) oral administration of the monoamine antidepressant agent; and
(ii) parenteral administration of the short-duration psychedelic agent.

7. The method for treating according to claim 2, wherein the psychiatric disorder is selected from a depressive disorder, an anxiety disorder, or both.

8. The method for treating according to claim 1, wherein the psychiatric disorder is:
a depressive disorder selected from major depressive disorder, persistent depressive disorder, bipolar disorder, bipolar depression, depression in terminally ill patients, and treatment resistant depression; or
an anxiety disorder selected from generalised anxiety disorder, phobia, panic disorder, social anxiety disorder, and post-traumatic stress disorder.

9. The method for treating according to claim 1, which further comprises:
a. assessing the patient with a baseline assessment prior to administration of the short-duration psychedelic agent using a rating scale and/or clinician reported outcomes and/or patient reported outcomes; and
b. assessing the patient with a post-dosing assessment following administration of the short-duration psychedelic agent.

10. The method for treating according to claim 9, wherein:
the psychiatric disorder is a depressive disorder and the baseline and post-dosing assessment is carried out using a depression rating scale selected from the Montgomery-Asberg Depression Rating Scale (MADRS), Hamilton Depression Rating Scale (HAMD17), Beck Depression Inventory-II, and combinations thereof, or
the psychiatric disorder is an anxiety disorder and the baseline and post-dosing assessment is carried out using a rating scale selected from the Beck Anxiety Inventory (BAI), Hamilton Anxiety Scale (HAM-A), State-Trait Anxiety Inventory (STAI-T), Generalised Anxiety Disorder Assessment (GAD-7), and combinations thereof.

11. The method for treating according to claim 1, wherein the administration of the short-duration psychedelic agent is parenteral administration selected from intravenous and intramuscular administration.

12. The method for treating according to claim 1, wherein a dose of the short-duration psychedelic agent is selected from the group consisting of:
about 20 to about 80 mg of N,N-dimethyltryptamine, or a pharmaceutically acceptable salt thereof, and
about 15 to about 80 mg of deuterated N,N-dimethyltryptamine, or a pharmaceutically acceptable salt thereof.

13. The method for treating according to claim 1, comprising the parenteral administration of a total dose of about 20 to about 23 mg of N,N-dimethyltryptamine or a pharmaceutically acceptable salt thereof, or deuterated N,N-dimethyltryptamine or a pharmaceutically acceptable salt thereof, or a pharmaceutically acceptable salt thereof, or a mixture thereof.

14. The method for treating according to claim 1, wherein a total dose of the short-duration psychedelic agent is about 15 to about 30 mg.

15. The method for treating according to claim 1, wherein the administration of the short-duration psychedelic agent is by intravenous infusion, and a total dose of the short-duration psychedelic agent is about 20 to about 23 mg.

16. The method for treating according to claim 1, wherein the short-duration psychedelic agent is a compound of formula I, or a pharmaceutically acceptable salt thereof: 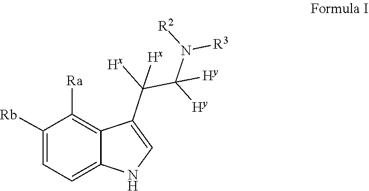
Ra is selected from H, and D;
Rb is selected from H and D;
R² and R³ are each independently selected from C(H^{z})₃; and
each H^{x}, H^{y} and H^{z} is independently selected from protium and deuterium.

17. The method for treating according to claim 16, wherein R² and R³ are both CH₃, or R² and R³ are both CD₃.

18. The method for treating according to claim 16, wherein each H^{x} is H.

19. The method for treating according to claim 16, wherein each H^{y} is H, or each H^{y} is D.

20. The method for treating according to claim 16, wherein each H^{x}, H^{y} and H^{z} is deuterium.

21. The method for treating according to claim 16, wherein the short-duration psychedelic agent is selected from the group consisting of: 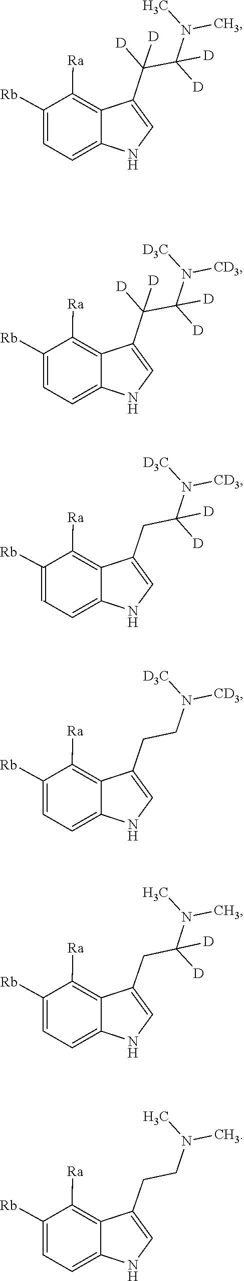
or a pharmaceutically acceptable salt thereof, or a mixture thereof.

22. The method for treating according to claim 21, wherein Ra is H.

23. The method for treating according to claim 21, wherein Rb is H.

24. The method for treating according to claim 16, wherein the compound is selected from:
N,N-dimethyltryptamine;
α,α-dideutero-N,N-dimethyltryptamine;
α,α,β,β-tetradeutero-N,N-dimethyltryptamine;
N,N-di(trideuteromethyl)tryptamine;
α,α-dideutero-N,N-di(trideuteromethyl)tryptamine;
α,α,β,β-tetradeutero-N,N-di(trideuteromethyl)tryptamine;
pharmaceutically acceptable salts thereof, and mixtures thereof.

25. The method for treating according to claim 1, comprising
(i) oral administration to the patient of a selective serotonin reuptake inhibitor over a period of 4 weeks or greater; and
(ii) parenteral administration to the patient of the short-duration psychedelic agent.

26. The method for treating according to claim 25, wherein the oral administration to the patient of the selective serotonin reuptake inhibitor is over a period of 6 weeks or greater.

27. The method for treating according to claim 1, wherein the treatment comprises psychedelic-assisted psychotherapy.

28. The method for treating according to claim 1, further comprising:
a. a Preparation Stage, comprising preparing the patient for a psychedelic experience;
b. an Administration Stage, comprising the administration of the short-duration psychedelic agent; and
c. an Integration Stage, comprising a psychiatrist or therapist led interview or discussion with the patient focused on the psychedelic experience.

29. A method of adjuvant treatment of a psychiatric disorder in a patient receiving treatment with a monoamine antidepressant agent, the method of adjuvant treatment comprising:
administering to the patient a short-duration psychedelic agent selected from N,N-dimethyltryptamine and pharmaceutically acceptable salts thereof, deuterated N,N-dimethyltryptamine and pharmaceutically acceptable salts thereof, and mixtures thereof, wherein the psychiatric disorder is at least one selected from a depressive disorder, an anxiety disorder, obsessive compulsive disorder, and an eating disorder; and the monoamine antidepressant agent is selected from a selective serotonin reuptake inhibitor and a serotonin-norepinephrine reuptake inhibitor.

30. The method of adjuvant treatment according to claim 29, wherein the short-duration psychedelic agent is administered to a patient who has received an adequate course of a monoamine antidepressant agent.

31. The method of adjuvant treatment according to claim 29, wherein the short-duration psychedelic agent is administered separately from the treatment with the monoamine antidepressant agent.

32. The method of adjuvant treatment according to claim 29, wherein the monoamine antidepressant agent is at least one selective serotonin reuptake inhibitor selected from citalopram, escitalopram, fluoxetine, fluvoxamine, paroxetine, sertraline, dapoxetine, and vortioxetine, and pharmaceutically acceptable salts thereof.
